(19) United States
(12) Patent Application Publication
(10) Pub. No.: US 2025/0277180 A1
(43) Pub. Date: Sep. 4, 2025

(54) APPARATUS, SYSTEMS AND METHODS FOR CONTINUOUS RNA MANUFACTURING

(71) Applicants: Arranta Bio Holdings, LLC, Watertown, MA (US); Massachusetts Institute of Technology, Cambridge, MA (US)

(72) Inventors: Edita Botonjic-Sehic, Watertown, MA (US); Syed Kaschif Ahmed, Watertown, MA (US); Hossein Hamedi, Watertown, MA (US); Marianna Sofman, Watertown, MA (US); Mark Jandreski, Watertown, MA (US); Aaron Cowley, Watertown, MA (US); Richard Braatz, Cambridge, MA (US); Allan Myerson, Cambridge, MA (US)

(21) Appl. No.: 19/066,510

(22) Filed: Feb. 28, 2025

Related U.S. Application Data
(60) Provisional application No. 63/560,143, filed on Mar. 1, 2024.

(51) Int. Cl. C12M 1/34; C12M 1/00; C12M 1/36

(52) U.S. Cl. CPC C12M 41/46; C12M 23/40; C12M 29/14; C12M 29/18; C12M 41/32; C12M 41/48; C12M 47/12

(57) ABSTRACT

The present invention provides apparatus, systems, and methods for continuous manufacturing of RNA utilizing in-line monitoring of the in vitro transcription reaction and related downstream processes, including chromatography and filtration processes.

### CROSS REFERENCE TO RELATED APPLICATIONS

This application claims priority to U.S. Provisional Application No. 63/560,143 filed Mar. 1, 2024, the contents of which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under contract 75F40122C00200 awarded by the Department of Health and Human Services. The Government has certain rights in the invention.

### BACKGROUND

Most biopharmaceuticals are manufactured using batch production methods in which human intervention is required to process a set quantity of material to be produced at the same time. Batch operations may require as long as 1-2 months or more from bioreactor to final formulated product. An alternative approach is continuous manufacturing which is attractive due to its potential to reduce costs while increasing productivity and improving product consistency. Continuous manufacturing processes have been developed in the chemical, petrochemical, food, and mechanical industries. In these contexts continuous processes have demonstrated less reliance on human labor and fewer gaps in transitioning between unit operations in the process resulting in increased productivity, while the smaller facility footprint required by a continuous process reduces facility costs.

There is a need for continuous manufacturing systems in the biopharmaceutical sector, as an alternative to the more time consuming, resource intensive, and expensive batch processes that represent the current standard of practice, as acknowledged by regulatory agencies which have urged the adoption of continuous biomanufacturing in this sector. See National Academies of Sciences, Engineering and Medicine. Continuous manufacturing for the modernization of pharmaceutical production. 2019.

While continuous bioprocessing has yet to be fully realized, several innovations in unit operations have made its implementation more feasible in biopharmaceutical manufacturing. In upstream processing these include developments in perfusion cell culture systems and continuous clarification systems such as continuous centrifugation, alternating tangential flow filtration, and acoustic wave separation. Developments in downstream operations include continuous chromatography and single-pass ultrafiltration and diafiltration capable of achieving high concentration factors and buffer exchange in a single pass of the process material through the module, e.g., in a continuous formulation process.

Despite these and other advances in adapting various unit operations to continuous processing, significant challenges remain in process integration, real-time monitoring and control systems. Currently, RNA manufacturing is performed in a series of batch processes including formation of the DNA template, in vitro transcription to produce the corresponding RNA molecules, a series of purification and concentration steps, lipid nanoparticle formulation, and final fill and finish of the RNA product. There is a need to develop systems and methods for continuous manufacture of RNA. This will embrace developing a fully integrated and continuous RNA process including adoption of novel process analytical technologies and data science techniques. The present invention addresses this need.

### BRIEF SUMMARY

The present invention provides modular apparatuses, including an in vitro transcription (IVT) apparatus, a tangential flow filtration (TFF) apparatus, and a chromatography apparatus which may be combined in various ways to provide a system for continuous manufacturing of RNA. Accordingly, also provided are related systems and methods.

In one aspect, provided is an apparatus including a reaction vessel configured for carrying out an RNA in vitro transcription (IVT) reaction, an IVT hold vessel configured to contain a volume of process fluid, one or more optical analytic instruments, and a CQA controller, where the apparatus is configured such that the IVT reaction vessel is fluidly connected via a first recirculation line to at least one optical analytic instrument and in fluid communication with the IVT hold vessel via a transfer line, the IVT hold vessel is fluidly connected via a second recirculation line to at least one optical analytic instrument and in fluid communication with a downstream unit apparatus via a second transfer line, and the CQA controller is configured to (i) receive data from the optical analytic instruments, (ii) monitor an amount of at least one reagent in the IVT reaction vessel and an amount of at least one of RNA product and impurities in the IVT hold vessel, (iii) determine whether a predetermined set of product quality attributes is satisfied, and (iv) execute a set of instructions to release the volume of process fluid from the IVT hold vessel to the downstream unit apparatus if the predetermined set of product quality attributes is satisfied. In aspects, the downstream unit apparatus is a tangential flow filtration (TFF) apparatus or a chromatography apparatus. The apparatus may also include where the one or more optical analytic instruments is selected from the group consisting of a Raman spectrometer, a UV-VIS spectrometer, a mid-infrared (IR) spectrometer, a near-IR spectrometer, and a refractometer. The apparatus may also include where the first and second recirculation lines comprise one or more inline sensors and/or flow cells. The apparatus may also include where the first and second transfer lines each comprise a pump and at least one valve configured to receive instructions from the CQA controller. The apparatus may also include where the IVT reaction vessel includes one or more inlet lines, each in fluid communication with a reservoir includes at least one reagent of the IVT reaction and each includes a pump and at least one valve, where the at least one valve is configured to receive instructions from the CQA controller. The apparatus may also include where the one or more optical analytic instruments is configured to measure an amount of one or more reagents of the IVT reaction. The apparatus may also include where the one or more optical analytic instruments is configured to measure an amount of RNA product in the IVT hold vessel. The apparatus may also include where the one or more inline flow cells is a UV variable pathlength flow cell, a Raman flow cell, a mid-IR flow cell, a near-IR flow cell, or an index of refraction flow cell. The apparatus may also include where the at least one valve is operated by a proportional-integral-derivative (PID) controller. The apparatus may also include where the one or more reagents of the IVT reaction is selected from nucleotides, plasmid DNA template, capping enzyme, RNA polymerase, pyrophosphatase enzyme, and magnesium. Other technical features may be readily apparent to one skilled in the art from the following figures, descriptions, and claims.

In aspects, the downstream unit apparatus is a TFF apparatus and the TFF apparatus includes a TFF filter unit in fluid communication with the IVT hold vessel and a TFF hold vessel, a TFF hold vessel in fluid communication with one or more optical analytic instruments via a recirculation line, where the TFF hold vessel is configured to contain a volume of process fluid, one or more optical analytic instruments includes at least one of an inline sensor or inline flow cell, and a CQA controller, where the apparatus is configured such that the TFF filter unit receives process fluid from the IVT hold vessel, separates RNA product into a product fluid stream, and transfers the product fluid stream to the TFF hold vessel where it recirculates through the at least one inline sensor or flow cell of the one or more optical analytic instruments, and the CQA controller is configured to (i) receive input from the optical analytic instruments, (ii) monitor at least one product attribute of the RNA product, (iii) determine whether a predetermined set of product quality attributes is satisfied, and (iv) execute a set of instructions to release the volume of process fluid from the TFF hold vessel to a downstream unit apparatus if the predetermined set of product quality attributes is satisfied.

In aspects, the downstream unit apparatus is a chromatography apparatus and the chromatography apparatus includes a chromatography unit in fluid communication with the IVT hold vessel and a chromatography hold vessel, a chromatography hold vessel in fluid communication with one or more optical analytic instruments via a recirculation line, where the chromatography hold vessel is configured to contain a volume of process fluid, one or more optical analytic instruments includes at least one of an inline sensor or inline flow cell, and a CQA controller, where the apparatus is configured such that the chromatography unit receives process fluid from the IVT hold vessel, separates RNA product into an eluate, and transfers the eluate to the chromatography hold vessel where it recirculates through the at least one inline sensor or flow cell of the one or more optical analytic instruments, and the CQA controller is configured to (i) receive input from the optical analytic instruments, (ii) monitor at least one product attribute of the RNA product, (iii) determine whether a predetermined set of product quality attributes is satisfied, and (iv) execute a set of instructions to release the volume of process fluid from the chromatography hold vessel to a downstream unit apparatus if the predetermined set of product quality attributes is satisfied.

In accordance with any of the foregoing aspects, the apparatus may also include where the one or more optical analytic instruments is selected from the group consisting of a Raman spectrometer, a UV-VIS spectrometer, a mid-infrared (IR) spectrometer, near-IR spectrometer, and a refractometer.

In accordance with any of the foregoing aspects, the apparatus may also include a TFF filter unit in fluid communication with and situated between the unit operation immediately downstream of the IVT reaction vessel, that is either the TFF unit or the chromatography unit, and the IVT reaction vessel. In accordance with these embodiments, the TFF filter unit is configured to receive a wash fluid from the chromatography unit and separate at least one reagent of the IVT reaction from the wash fluid and return the at least one reagent to the IVT reaction vessel. In aspects, the at least one reagent comprises one or more nucleotides, plasmid DNA template, capping enzyme, RNA polymerase, and/or pyrophosphatase enzyme for recycling back to the IVT reaction vessel.

In aspects, a TFF apparatus is situated downstream of the chromatography apparatus and configured to receive process fluid from the chromatography hold vessel, where the TFF apparatus includes a TFF filter unit in fluid communication with the chromatography hold vessel and a TFF hold vessel, a TFF hold vessel in fluid communication with one or more optical analytic instruments via a recirculation line, where the TFF hold vessel is configured to contain a volume of process fluid, one or more optical analytic instruments including at least one of an inline sensor or inline flow cell, and a CQA controller, where the apparatus is configured such that the TFF filter unit receives process fluid from the chromatography hold vessel, separates RNA product into a product fluid stream, and transfers the product fluid stream to the TFF hold vessel where it recirculates through the at least one inline sensor or flow cell of the one or more optical analytic instruments, and the CQA controller is configured to (i) receive input from the optical analytic instruments, (ii) monitor at least one product attribute of the RNA product in the TFF hold vessel, (iii) determine whether a predetermined set of product quality attributes is satisfied, and (iv) execute a set of instructions to release the volume of process fluid from the TFF hold vessel to a downstream unit apparatus if the predetermined set of product quality attributes is satisfied.

In aspects, a second TFF apparatus may be situated between the IVT hold vessel and the chromatography apparatus and configured to receive process fluid from the IVT hold vessel, the second TFF apparatus including a TFF filter unit in fluid communication with the IVT hold vessel and a TFF hold vessel, a TFF hold vessel in fluid communication with one or more optical analytic instruments via a recirculation line, where the TFF hold vessel is configured to contain a volume of process fluid, one or more optical analytic instruments including at least one of an inline sensor or inline flow cell, and a CQA controller, where the apparatus is configured such that the TFF filter unit receives process fluid from the IVT hold vessel, separates RNA product into a product fluid stream, and transfers the product fluid stream to the TFF hold vessel where it recirculates through the at least one inline sensor or flow cell of the one or more optical analytic instruments, and the CQA controller is configured to (i) receive input from the optical analytic instruments, (ii) monitor at least one product attribute of the RNA product in the TFF hold vessel, (iii) determine whether a predetermined set of product quality attributes is satisfied, and (iv) execute a set of instructions to release the volume of process fluid from the TFF hold vessel to the chromatography unit if the predetermined set of product quality attributes is satisfied. Other technical features may be readily apparent to one skilled in the art from the following figures, descriptions, and claims.

In accordance with any of the foregoing aspects, the one or more optical analytic instruments includes a flow cell for inline sampling of the process fluid. In aspects, the CQA controller performs chemometrics utilizing offline analytics. In aspects, included is a digital twin operably connected to each of the CQA controllers. In aspects, the digital twin controls the system, which includes each of the unit apparatuses. In aspects, the one or more optical analytic instruments includes a Raman spectrometer a mid-IR spectrometer and/or a UV-VIS spectrometer configured to measure a concentration of RNA product in one or more of the IVT reaction vessel, the IVT hold vessel, a TFF hold vessel, or a chromatography hold vessel. In aspects, the one or more optical analytic instruments includes a Raman spectrometer configured to measure a concentration of impurities in the RNA product in one or more of the IVT reaction vessel, the IVT hold vessel, a TFF hold vessel, or a chromatography hold vessel. In aspects, the predetermined set of product quality attributes includes a concentration of RNA product and a percentage purity of the RNA. Other technical features may be readily apparent to one skilled in the art from the following figures, descriptions, and claims.

Also provided are systems comprising an IVT apparatus as described herein fluidly connected to one or more TFF apparatuses and/or one or more a chromatography apparatuses as described here. In aspects, the system includes a TFF filter unit in fluid communication with and situated between the chromatography unit and the IVT reaction vessel. In aspects, the TFF filter unit is configured to receive wash fluid from the chromatography unit, separate at least one reagent of the IVT reaction and return the at least one reagent to the IVT reaction vessel.

Also provided are methods for continuous RNA manufacturing. In an aspect, the method includes contacting an initial amount of a linear plasmid DNA template with a reaction mixture in an in vitro transcription (IVT) reaction vessel under conditions suitable for a IVT reaction, monitoring progression of the IVT reaction via data received by a CQA controller from one or more optical analytic instruments in fluid communication with the IVT reaction vessel via a recirculation line, determining, by a first CQA controller, that the reaction has reached a predetermined state, executing, by the first CQA controller, a set of instructions to release a volume of process fluid from the IVT reaction vessel to an IVT hold vessel, receiving, by a second CQA controller, data from one or more optical analytic instruments in fluid communication with the IVT hold vessel via a recirculation line, determining, by the second CQA controller, whether RNA product has reached a predetermined level and percentage purity in the IVT hold vessel, and executing, by the second CQA controller, a set of instructions to release a volume of process fluid from the IVT hold vessel to a downstream unit apparatus if the RNA product has reached the predetermined level and purity. The method may also include where the downstream unit apparatus is a tangential flow filtration (TFF) apparatus as described above or infra. The method may also include where the downstream unit apparatus is a chromatography apparatus as described above or infra. The method may also include where the CQA controllers perform chemometrics utilizing offline analytics. In aspects, the CQA controllers are controlled by operation of a digital twin. Other technical features may be readily apparent to one skilled in the art from the following figures, descriptions, and claims.

In aspects, the method includes a first tangential flow filtration (TFF) apparatus upstream of the chromatography apparatus and a second TFF apparatus downstream of the chromatography apparatus. The method may also include where the method includes recovering at least one reagent of the IVT reaction mixture and recycling the at least one reagent to the IVT reaction vessel via operation of a TFF unit in fluid communication with and situated between either the downstream TFF apparatus or the downstream chromatography apparatus and the IVT reaction vessel. The method may also include where the at least one reagent includes one or more nucleotides, plasmid DNA template, capping enzyme, RNA polymerase, and/or pyrophosphatase enzyme. The method may also include where the one or more nucleotides include guanosine-5′-triphosphate, adenosine triphosphate, cytidine triphosphate, uridine triphosphate, pseudouridine triphosphate, dihydrouridine triphosphate, 4-thiouridine, inosine triphosphate, 7-methylguanosine triphosphate, 2,7-dimethylguanosine triphosphate, and/or 2,2,7-trimethylguanosine triphosphate. Other technical features may be readily apparent to one skilled in the art from the following figures, descriptions, and claims.

In one aspect, provided is a method for continuous RNA manufacturing, where the method includes acquiring a series of Raman spectra of process fluid of an in vitro transcription (IVT) reaction via an in-line Raman flow cell in fluid communication with an IVT reaction vessel, transforming the Raman spectra into IVT Critical Quality Attribute (CQA) data, monitoring progression of the IVT reaction via the CQA data, determining, based on the IVT CQA data, whether the IVT reaction has reached a predetermined state, and executing, by a CQA controller, a set of instructions to release a volume of process fluid from the IVT reaction vessel to an IVT hold vessel if the IVT reaction has reacted a predetermined state. The method may also include where the transforming includes normalizing the Raman spectra and determining an intensity at wavelengths between 800-1200 cm⁻¹. In aspects, the wavelengths include one or more of 812 cm⁻¹, 990 cm⁻¹ and 1122 cm⁻¹. The method may also include where the CQA data is mRNA concentration data. The method may also include where the CQA data is NTP concentration data. The method may also include acquiring spectral data from a second optical analytic instrument in fluid communication with the IVT hold vessel via an in-line flow cell situated in a recirculation line, transforming the spectral data into Product Critical Quality Attribute (CQA) data, determining, by a second CQA controller, whether RNA product has reached a predetermined concentration and percentage purity in the IVT hold vessel based on the Product CQA data, and executing, by the second CQA controller, a set of instructions to release a volume of process fluid from the IVT hold vessel to a downstream unit apparatus if the RNA product has reached the predetermined level and purity. Other technical features may be readily apparent to one skilled in the art from the following figures, descriptions, and claims.

### BRIEF DESCRIPTION OF THE SEVERAL VIEWS OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers, with redundant description omitted. For purposes of clarity and simplicity, not every element is labeled in every figure, nor is every element of each embodiment shown where illustration is not necessary to allow those of ordinary skill in the art to understand the disclosure.

To easily identify the discussion of any particular element or act, the most significant digit or digits in a reference number refer to the figure number in which that element is first introduced.

FIG. 1 is a diagram of an IVT unit apparatus in accordance with one embodiment.

FIG. 2 is a diagram of the IVT unit apparatus of FIG. 1 including a digital twin.

FIG. 3 is a line graph illustrating the fitting of Raman data to an exponential growth function for RNA product and an Ordinary Differential Equation (ODE) function for nucleotides ATP, CTP, GTP, and UTP.

FIG. 4 is a schematic illustration of a self-regulated IVT unit apparatus in accordance with one embodiment.

FIG. 5A is a diagram of an IVT unit apparatus in accordance with one embodiment illustrating connections between optical analytic instruments (Raman and ultraviolet (UV) spectrometers), at least one pump, at least one stir tank reactor (STR) and at least one feed vessel.

FIG. 5B is a diagram showing a TFF unit connected to the IVT unit apparatus of FIG. 5A to provide an IVT and TFF system in accordance with one embodiment.

FIG. 6A is a diagram showing a chromatography unit connected to the IVT unit apparatus of FIG. 5A to provide an IVT and chromatography system in accordance with one embodiment.

FIG. 6B illustrates an aspect of an IVT and chromatography system where certain reagents of the IVT reaction, for example one or more of nucleotides, plasmid DNA template, capping enzyme, RNA polymerase, and pyrophosphatase enzyme are recovered through a TFF unit and recycled back into the IVT reaction vessel.

FIG. 7 is a diagram of an IVT system including an IVT unit apparatus, a chromatography unit apparatus, and a TFF unit apparatus. Also pictured is an optional TFF unit fluidly connected between the chromatography unit and the IVT reaction vessel for recycling of IVT reaction reagents.

FIG. 8 is a diagram of an IVT system including an IVT unit apparatus, a first TFF unit apparatus, a chromatography unit apparatus, and a second TFF unit apparatus. Also pictured is an optional TFF unit fluidly connected between the chromatography unit and the IVT reaction vessel for recycling of IVT reaction reagents.

FIG. 9 is a diagram illustrating the flow of data in a single unit operation, in this case represented by the IVT operation.

FIG. 10A is a line graph representing the process of calibrating a data driven model using hundreds of Raman spectra measurements mapped into reference values determined by offline analytics. Model coefficients determined from the process of calibration in FIG. 10A are used to estimate RNA concentration based on a new Raman spectra in real-time.

FIG. 10B illustrates an aspect of the subject matter in accordance with one embodiment.

FIG. 11 is a line graph illustrating in vitro transcription univariate analysis.

FIG. 12A is a line graph showing partial least square regression between the model predictions and reference measurements.

FIG. 12B is a line graph showing model prediction for mRNA concentration for an independent data set (acquired from a separate run).

FIG. 13 is a schematic illustration of the process of modeling risk assessment and generic model development.

FIG. 14 is a schematic illustrating the importance of data flow and management.

FIG. 15 is a graph illustrating a distribution of samples according to the mRNA concentration of the samples.

FIG. 16 is a line graph of raw spectral data acquired from IVT runs using a in-line Raman flow cell.

FIG. 17 is a line graph of a normalized 2^{nd} derivative of spectral data acquired from IVT runs using in-line Raman flow cell.

FIG. 18 is a regression plot for model training illustrating that the model predictions correlates to the off-line measurements.

FIG. 19 is a regression plot for model validation illustrating that the model prediction correlates to the off-line concentration.

FIG. 20 is a line graph illustrating model predictions for the validation dataset run CP1-Fluc-007.

FIG. 21 is a line graph illustrating model predictions for the validation dataset run CP1-Covid-009.

FIG. 22 is a line graph illustrating model predictions for the validation dataset run CP1-Fluc-003.

FIG. 23 is a line graph of PLS model loading vector #1 vs mRNA Raman spectrum illustrating specificity of the model to mRNA.

FIG. 24 is a line graph of raw spectral data acquired from IVT during CPC-Fluc-003 run using in-line Raman flow cell.

FIG. 25 is a line graph of normalized spectral data acquired from IVT during CPC-Fluc-003 run using in-line Raman flow cell.

FIG. 26 is a line graph of normalized spectral data acquired from IVT during CPC-Fluc-003 run using in-line Raman flow cell.

FIG. 27 is a graph illustrating Raman intensity at 812 cm⁻¹ as a function of transcription time.

FIG. 28 is a graph illustrating Raman intensity at 1122 cm⁻¹ as a function of transcription time.

FIG. 29 is a graph illustrating Raman intensity at 990 cm⁻¹ as a function of transcription time.

FIG. 30 is a graph illustrating Raman spectra of NTPs obtained by principal component analysis (PCA).

FIG. 31 is a line graph of raw spectral data acquired from IVT runs using in-line Raman flow cell.

FIG. 32 is a graph illustrating n normalized 2^{nd} derivative of spectral data acquired from IVT runs using in-line Raman flow cell.

FIG. 33 is a regression plot for training of the PLS model predicting CTP.

FIG. 34 is a regression plot for validation of the PLS model predicting GTP.

FIG. 35 is a regression plot for training of the PLS model predicting GTP.

FIG. 36 is a regression plot for validation of the PLS model predicting GTP.

FIG. 37 is a regression plot for training of the PLS model predicting ATP.

FIG. 38 is a regression plot for validation of the PLS model predicting ATP.

### DETAILED DESCRIPTION

The present invention provides modular apparatuses, including an in vitro transcription (IVT) apparatus, a tangential flow filtration (TFF) apparatus, and a chromatography apparatus which may be combined to provide a system for continuous manufacturing of RNA. Continuous manufacturing is attained by closed loop control of unit operations by each apparatus. This is accomplished in part by the provision of inline Process Analytical Technologies (PAT) tools in operable communication with a Critical Quality Attributes (CQA) soft sensor control module, or “CQA controller” as part of each apparatus.

The term “CQA controller” refers to a control system that includes PAT management software, data modeling software, and a process control system (PCS) which may be in the form of a Supervisory Control and Data Acquisition (SCADA) system. The CQA controller may comprise one or more predictive data models, including univariate and multivariate models. It is contemplated that the models are initially calibrated using offline analytics as described in more detail infra. In aspects, the CQA controller may include algorithms for carrying out one or more statistical methods including multiple linear regression (MLR), partial least squares regression (PLS), and structured additive regression (STAR). In aspects, a CQA controller as described herein may also include one or more models based on random forest (RF), support vector machine regression (SVM), a neural network (NN), a deep learning (DL) algorithm, and Gaussian process regression (GPR). The CQA controller may also perform one or more chemometric operations. Chemometric operations may include one or more of principal component analysis (PCA), partial least squares (PLS) regression, cluster analysis, discriminant analysis, multivariate curve resolution (MCR), and outlier detection.

In operation, the CQA controller continuously monitors and adjusts process parameters of the unit apparatus, e.g., the IVT apparatus, the TFF apparatus, or the chromatography apparatus, based on real-time data provided by inline sensors and/or flow cells of one or more optical analytic instruments. In this manner, the CQA controller maintains desired conditions and optimizes the unit process, whether it be the IVT reaction or a downstream TFF or chromatography operation. Also provided is a digital twin operably connected to each CQA controller in the system. The digital twin incorporates data-driven modeling as well as mechanistic modelling to model and predict values of process variables and process parameters for the various unit operations and provides control of the entire manufacturing process.

Accordingly, in one aspect provided is an in vitro transcription (IVT) apparatus including an IVT reaction vessel, an IVT hold vessel, one or more optical analytic instruments, and a CQA controller that provides for closed loop control of the in vitro transcription process.

Also provided is a TFF apparatus including a TFF unit, a TFF hold vessel, one or more optical analytic instruments, and a CQA controller that provides for closed loop control of the TFF process.

Also provided is a chromatography apparatus including a chromatography unit, a chromatography hold vessel, one or more optical analytic instruments, and a CQA controller that provides for closed loop control of the chromatography process.

Each of the IVT, TFF, and chromatography apparatuses includes a recirculation line providing fluid communication between the hold vessel and the one or more optical analytic instruments. Included are one or more of inline sensors and/or flow cells to provide for continuous measurement of process and product attributes by the analytic instruments which transmit process and product data to the CQA controller. For the IVT apparatus, there is an additional recirculation line providing fluid communication between the IVT reaction vessel and one or more optical analytic instruments to provide for monitoring of the IVT reaction. In operation, the CQA controller determines whether a volume of process fluid in the hold vessel is rejected or released to a downstream unit operation, for example a downstream tangential flow filtration (TFF) apparatus or chromatography apparatus, by determining whether a predetermined set of process and/or product quality attributes is satisfied, and executing a set of instructions to release the volume of process fluid from the hold vessel to the downstream apparatus where the predetermined set of product quality attributes is satisfied.

Each of the IVT, TFF, and chromatography apparatuses includes transfer lines in fluid communication with and situated between the unit apparatus, i.e., the IVT reaction vessel, the TFF unit, or the chromatography unit, and the hold vessel as well as between the hold vessel and a downstream unit apparatus. The transfer lines may include a pump and at least one valve configured to receive instructions from the CQA controller. In aspects, the at least one valve is operated by a proportional-integral-derivative (PID) controller.

In aspects, the one or more optical analytic instruments includes one or more of a Raman spectrometer, a UV-VIS spectrometer, a mid-infrared (IR) spectrometer, a near-IR spectrometer and a refractometer. The optical analytic instruments are fluidly connected via recirculation lines with the vessels holding the process fluid. The recirculation lines comprise one or more inline sensors and/or flow cells. In aspects, the one or more inline flow cell is a UV variable pathlength flow cell, a Raman flow cell, a mid-IR flow cell, a near-IR flow cell, or an index of refraction flow cell.

In aspects, the IVT apparatus includes a first Raman and a first UV-VIS spectrophotometer in fluid communication with the IVT reaction vessel via a recirculation line and a second Raman and a second UV-VIS spectrophotometer in fluid communication with the IVT hold vessel via a second recirculation line. The Raman and UV-VIS spectrophotometers are configured to provide data to a CQA controller for the IVT reaction. In aspects, the data includes measurements to determine an amount of at least one of RNA product and impurities in the IVT reaction vessel and/or the IVT hold vessel. In aspects, the data includes measurements to determine an amount of one or more reagents in the IVT reaction vessel, such as the amount of nucleotide triphosphates (NTPs), linear plasmid DNA template, RNA polymerase, magnesium ions, and/or capping enzyme. In aspects, the data may also include pH, temperature, conductivity, pressure, and/or UV data obtained from the IVT reaction vessel. The NTPs may include any combination of adenosine triphosphate (ATP), cytidine triphosphate (CTP), guanosine triphosphate (GTP), uridine triphosphate (UTP) and pseudo UTP. In aspects, the NTPs may include any combination of guanosine-5′-triphosphate, adenosine triphosphate, cytidine triphosphate, uridine triphosphate, pseudouridine triphosphate, dihydrouridine triphosphate, 4-thiouridine, inosine triphosphate, 7-methylguanosine triphosphate, 2,7-dimethylguanosine triphosphate, and 2,2,7-trimethylguanosine triphosphate.

In aspects, the IVT reaction vessel may also include one or more inlet lines, each in fluid communication with a reservoir comprising at least one reagent of the IVT reaction. In some aspects, fluid is moved through the inlet lines by action of a pump in coordination with at least one valve, wherein the pump and at least one valve are configured to receive instructions from the CQA controller. In aspects, the at least one valve is operated by a proportional-integral-derivative (PID) controller. In aspects, the IVT reaction vessel is in fluid communication with one or more of a buffer reservoir, an NTP reservoir, a linear plasmid DNA reservoir, a magnesium ion reservoir, a capping agent reservoir, and an RNA polymerase reservoir.

Accordingly, also provided is a system for continuous RNA manufacturing comprising an IVT apparatus as described herein fluidly connected to one or more TFF apparatuses and/or chromatography apparatuses. The modular IVT, TFF, and chromatography apparatuses provide for real-time release testing (RTRT) at any of several critical control points (CCPs) in the system. The term real-time release testing is used in its ordinary and customary manner to refer to in-process evaluation of the process stream containing the RNA product based on process data, including a combination of measured material attributes provided by the optical analytic instruments and the models of the CQA controller.

In aspects, the system may also comprise one or more of a perfusion bioreactor, simulated moving bed chromatography, liquid-liquid extraction, swing-chromatography, and continuous liquid injection for nanoparticle formation.

Also provided are methods for real-time monitoring, testing, and release of a product stream at defined points in an RNA manufacturing process using the apparatuses and systems described herein.

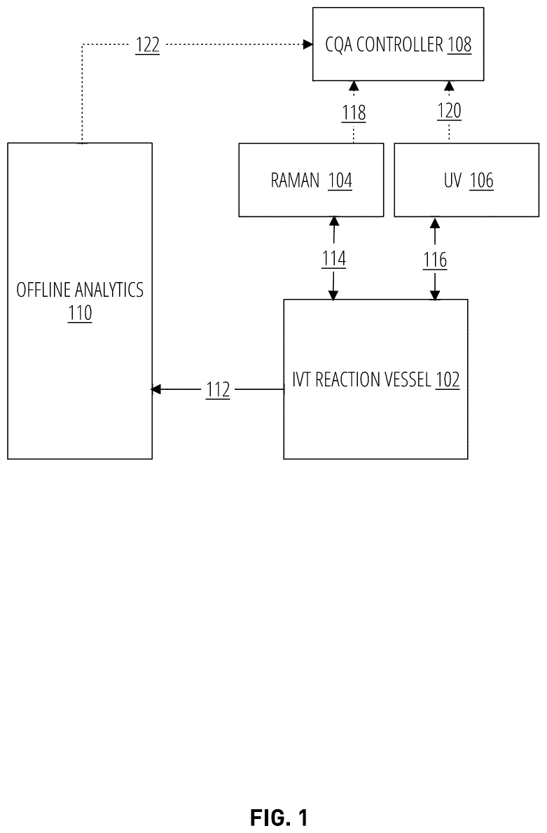

Turning to the drawings, FIG. 1 is a schematic of an IVT unit apparatus in accordance with one embodiment. Shown is an IVT reaction vessel 102 in which the in vitro transcription reaction takes place. The IVT reaction vessel 102 may be any suitable vessel for conducting an in vitro transcription reaction. In aspects, the IVT reaction vessel 102 is a stirred tank bioreactor. As shown, the IVT reaction vessel 102 is fluidly connected with two optical analytic instruments, a Raman 104 spectrometer and a UV 106 spectrometer. The solid double arrows indicate a fluid recirculation loop between the IVT reaction vessel 102 and each of the optical analytic instruments. In use, measurements are taken via in-line or at-line flow cells. In aspects, the UV in-line or at-line flow cell may be a variable pathlength flow cell.

In operation, the CQA controller 108 monitors the kinetics of the in vitro transcription reaction, for example by analyzing data from the optical analytic instruments which may include measurements for determining the concentration of nucleotide triphosphates (NTPs) and/or other reactants and cofactors, for example the concentration of linear template plasmid DNA (pDNA), magnesium ions (Mg), ribonucleic acid (RNA) polymerase, and inorganic pyrophosphatase (iPPS). The optical analytic instruments may also perform measurements for determining the concentration of newly synthesized RNA product as the reaction progresses as well as other characteristics of the RNA product and/or the presence of impurities in the product stream.

It is also contemplated that a volume of process fluid may be removed from the IVT reaction vessel 102 for offline analytics 110. In the figure, dotted lines indicate flow of data 118, 120, and 122 from the optical analytic instruments and the offline analytics 110 to the CQA controller 108. Offline analytics may include spectroscopy, chromatography, electrophoresis, multiangle light scattering, or fluorescence analysis. In aspects, the offline analytics may include one or more of UV-VIS spectroscopy, infrared (IR) spectroscopy, high-performance liquid chromatography (HPLC), mass spectroscopy (MS), capillary electrophoresis, a quantitative polymerase chain reaction (qPCR), Fluorescence-Based Solution Assay, and/or Multiangle Light Scattering (MALS). The offline analytics 110 are used to calibrate the CQA controller 108. Reference samples are used to generate appropriate models based on the underlying data and assumptions for a given unit operation, such as an IVT process, a chromatographic separation process, or a tangential flow filtration (TFF) process. The model may be a mechanistic model or a data-driven model, or a hybrid model that combines aspects of both mechanistic and data-driven models. A mechanistic model refers to a model based on biophysical relationships that have been mathematically elucidated based on a full mathematical understanding of the process and may also be referred to as “white-box models”. Data-driven models are mathematical models based solely on the statistical relationships between data, primarily data obtained or derived from online sensors and offline analysis. Data-driven models are not based on biophysical relationships and may also be referred to as “black-box models.” Hybrid models combine mechanistic and data-driven models.

Offline analytics may also be used to calibrate the CQA controllers of a TFF apparatus and a chromatography apparatus, as described herein, for example during a development process. In general, the CQA controllers of the unit apparatuses described herein may include algorithms to measure the relationship between variables. Generally the methods include a correlation analysis to establish correlations between sensor signals, process parameters, and quantity and quality parameters, some of which may be measured offline. For example, the extent of the linear relationship may be determined using a Pearson's correlation. Other methods are available to measure nonlinear relationships, for example, Spearman's rank correlation, which is a nonparametric measure of rank correlation reporting the statistical relationship between the rankings of two variables. In aspects, the CQA controllers of the unit apparatuses described herein may include algorithms for carrying out one or more statistical methods including multiple linear regression (MLR), partial least squares regression (PLS), and structured additive regression (STAR). In aspects, a CQA controller as described herein may also include one or more models based on random forest (RF), support vector machine regression (SVM), neural networks (NNs), deep learning (DL), and Gaussian process regression (GPR).

In a particular implementation, the CQA controllers utilize PLS models calibrated using the offline analytical measurements. In a further specific implementation, the PLS models use principal component analysis (PCA) to identify the parameters contributing the most variation in the Raman spectral data (X space) which is correlated to the offline reference values (e.g, HPLC measurements (Y space)).

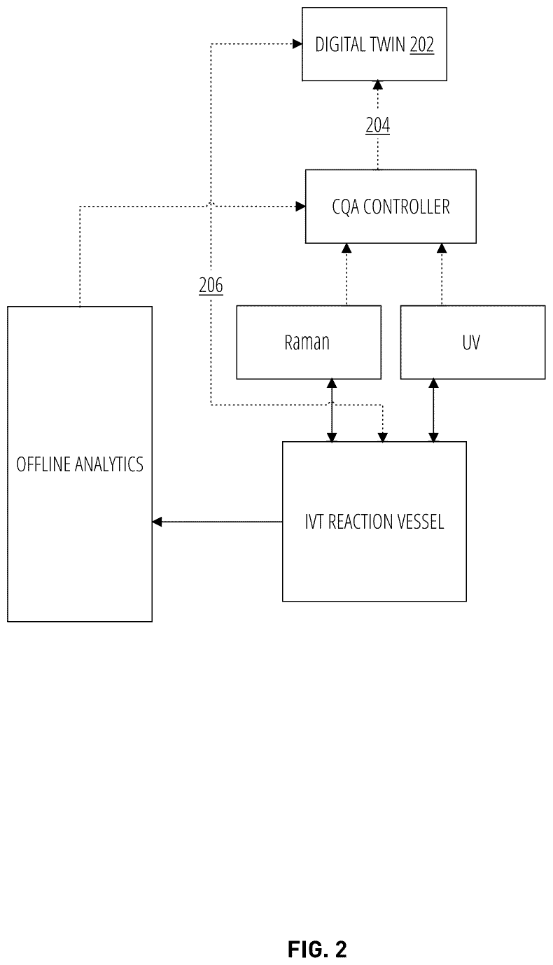

FIG. 2 illustrates an IVT unit apparatus as described in FIG. 1 where the apparatus is controlled by a digital twin 202. The IVT digital twin incorporates data-driven modeling as well as mechanistic modelling to model and predict values of process variables and process parameters for the in vitro transcription reaction. In aspects, the mechanistic modelling may include differential equations for kinetic processes. For example, a series of ordinary differential equations with fitted parameters and initial conditions may be utilized to predict certain variables of the reaction including concentration of NTPs, plasmid DNA (pDNA), magnesium (Mg), and RNA as well as solution pH and temperature (T). The hybrid modelling approach described here allows powerful machine learning tools to account for unknown phenomena when combined with mechanistic models that better describe the biological system and reaction processes. Data-driven modelling can utilize historical and newly collected data from the process runs in order to make predictions on certain attributes such as yield and purity. Machine learning-driven algorithms, e.g., linear regression, partial least squares regression (PLS), random forest, and decision trees, may be implemented to develop the models and make predictions.

In specific implementations, the models provide an understanding of NTP consumption and mRNA production rates during the in vitro transcription reaction, which is utilized to determine optimal starting concentrations of the reaction components.

In a specific implementation of mechanistic modelling, the series of differential equations used to predict variables of the in vitro transcription reaction may be written as:

$$\frac{dNTP}{dt} = α {f}_{1} ( NTP , xRNA , t , Raman , FlowVPX , Mg , pDNA , PPi , pH , T )$$

$$\frac{dxRNA}{dt} = β {f}_{2} ( NTP , xRNA , t , Raman , FlowVPX , Mg , pDNA , PPi , pH , T )$$

$$\frac{dMg}{dt} = γ {f}_{3} ( NTP , xRNA , t , Raman , FlowVPX , Mg , pDNA , PPi , pH , T )$$

$$\frac{dpDNA}{dt} = δ {f}_{4} ( NTP , xRNA , t , Raman , FlowVPX , Mg , pDNA , PPi , pH , T )$$

$$\frac{dPPi}{dt} = ε {f}_{5} ( NTP , xRNA , t , Raman , FlowVPX , Mg , pDNA , PPi , pH , T )$$

$$\frac{dpH}{dt} = ζ {f}_{6} ( NTP , xRNA , t , Raman , FlowVPX , Mg , pDNA , PPi , pH , T )$$

$$\frac{dT}{dt} = η {f}_{7} ( NTP , xRNA , t , Raman , FlowVPX , Mg , pDNA , PPi , pH , T )$$

The set of parameters α, β, γ, δ, ε, etc., may be fitted to the soft-sensor data, offline analytics, and in-process data.

The mechanistic models may be further refined using fitted equations with known analytical values to describe nonlinear relationships between various quantities. In the rate equations depicted through the ordinary differential equations, Michaelis-Menten equations may be used to describe the enzyme-catalyzed in vitro transcription reaction. In addition, power law equations, including exponential growth and decay functions, may be used to describe the functional relationship between the different variables.

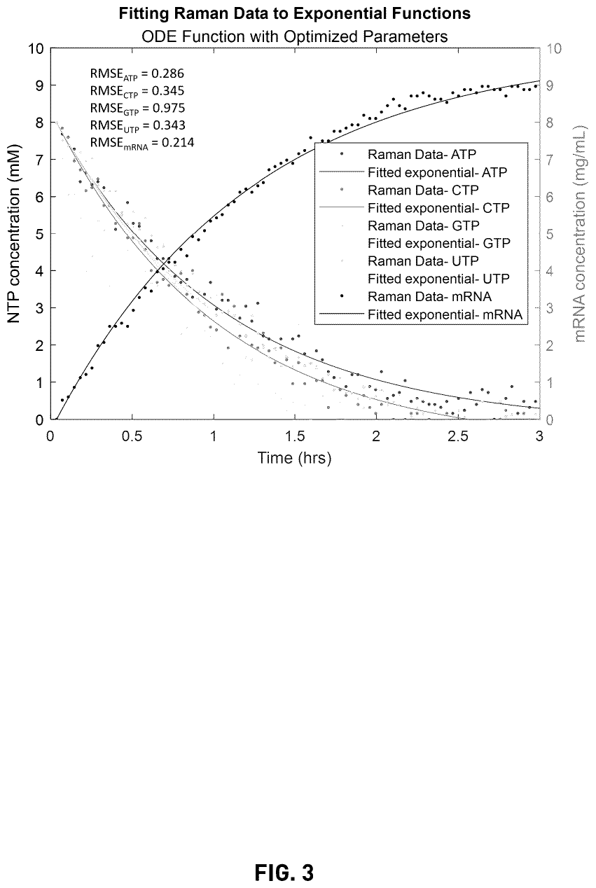

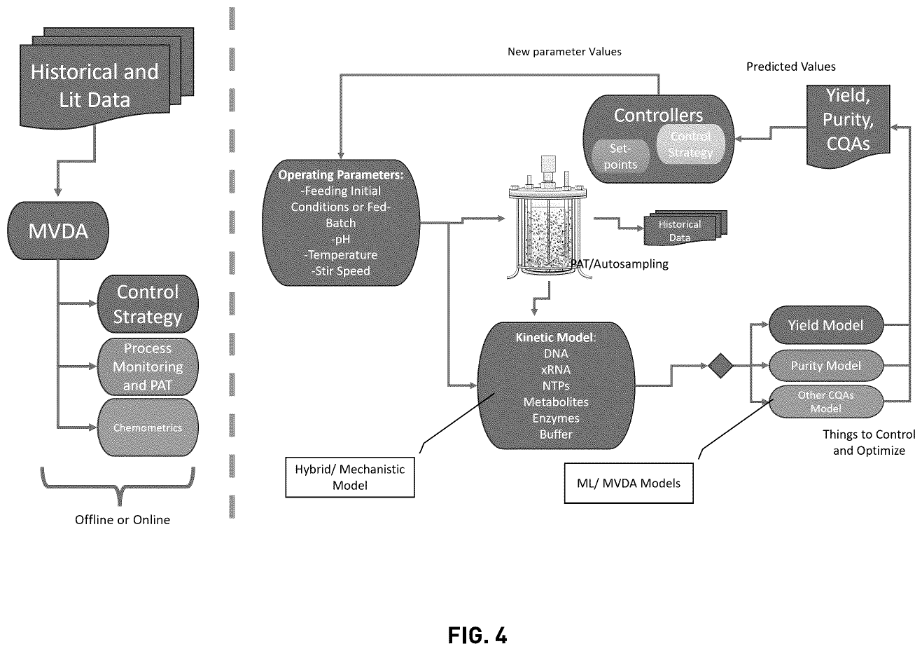

FIG. 3 shows preliminary results of a soft-sensor calibrated with offline analytics to model the in vitro transcription reaction kinetics. The initial reaction mixture is represented by 8 mM of each NTP (ATP, CTP, GTP, UTP). As the reaction continues forward, the NTPs are consumed as the RNA is produced. The final RNA concentration is confirmed with offline analytics using standard UV methods. A non-linear curve fitting algorithm such as lsqcurvefit is used to fit the Raman spectral data to an exponential growth and decay function for the NTP and RNA concentrations, respectively. The initial conditions and offline analytics data are used to adjust the final fit of the reaction functions. This information is utilized for advanced process control (APC), end-point control, and prediction of further process conditions as illustrated in FIG. 4 and to calibrate the IVT CQA controller. The dotted data represent normalized Raman trending for mRNA and NTP concentration during the reaction. The lines are the fitting results. The model predicts how each NTP is consumed and when RNA concentration reaches a plateau. Root mean square of errors (RMSE) are shown on the plot for each fitting.

FIG. 4 is a schematic illustration of a self-regulated IVT apparatus in accordance with one embodiment. The figure illustrates how modelling is utilized to optimize operating parameters.

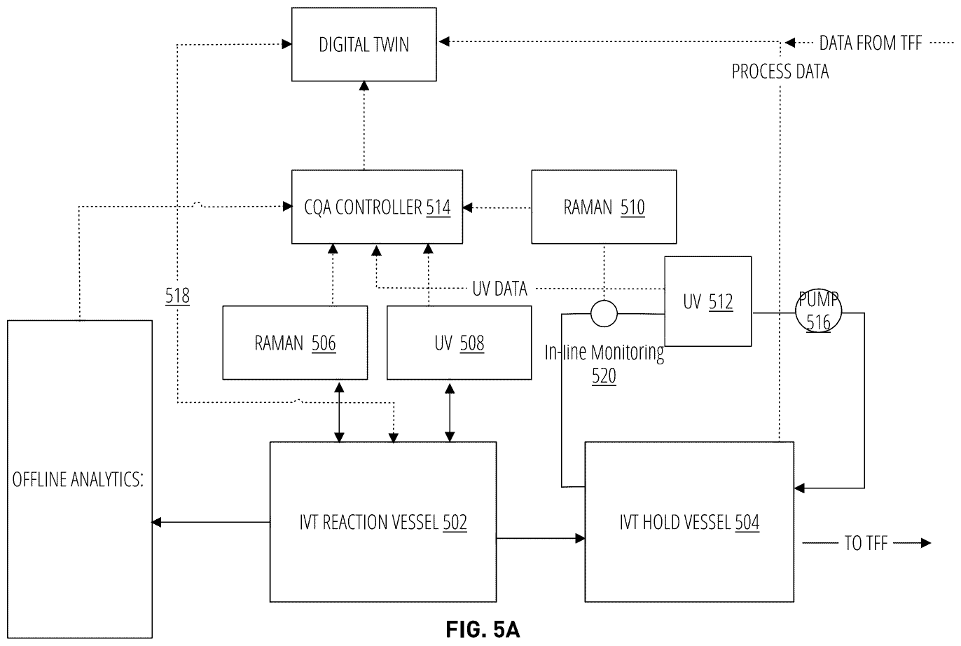

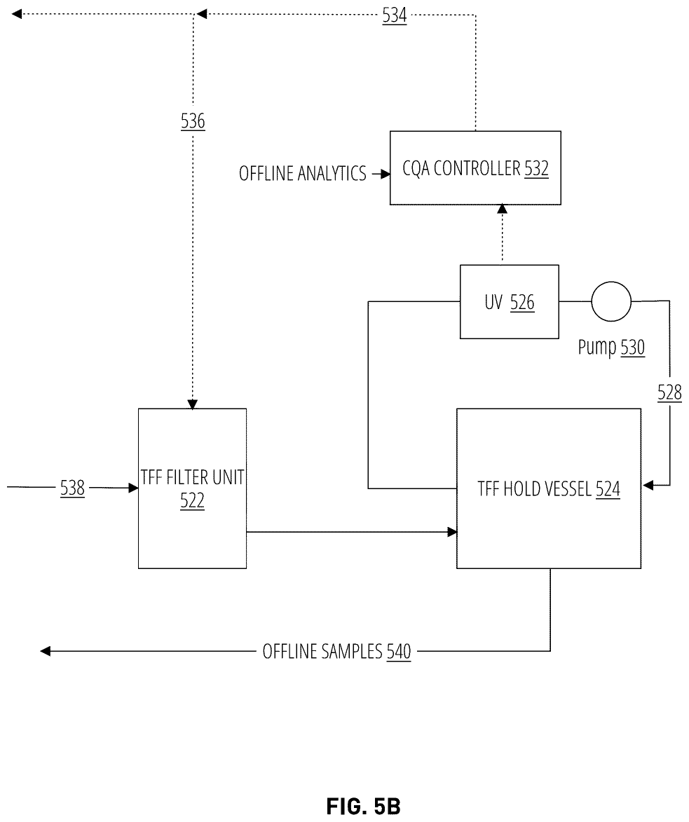

FIG. 5A illustrates an IVT unit apparatus in accordance with an embodiment where the unit apparatus as described in FIG. 2 includes an IVT hold vessel 504 fluidly connected in a recirculation loop with a second set of optical analytic instruments, a second Raman 510 and a second UV 512 spectrometer. In operation, a volume of RNA product containing process fluid is transferred, via operation of a pump in coordination with at least one valve (not shown), from the IVT reaction vessel 502 to the IVT hold vessel 504 where it is recirculated through inline or at-line flow cells connected to the second set of optical analytic instruments. These additional post-IVT reaction analytic instruments monitor the concentration of product RNA and critical quality attributes (CQAs) such as RNA integrity and purity. RNA integrity is a quality metric assessing the size and length of the RNA product and is used to confirm that the product comprises the full length of the intended polynucleotide sequence. Off-line analytics are utilized for calibration during model development. For RNA integrity, the offline analytic measurements may be performed, for example, using a Fragment Analyzer to determine the size and length of the RNA product. The concentration of remaining reaction components and specific impurities may also be monitored at this point in the process. The data, indicated in the figure by dotted lines, is transmitted to the CQA controller 532 and the digital twin 202.

Thus, in operation, the IVT apparatus provides a combination of sensors and control strategies for real-time release testing (RTRT) of a volume of process fluid in the IVT hold vessel 504. If the CQAs are met, the system releases the process fluid to a downstream unit operation, such as a TFF or chromatography unit operation. This approach also provides for release of a volume of process fluid after a unit operation is complete without the use of offline analytics during processing. Instead, offline analytics are used in a development phase to calibrate the CQA controllers, as well as for model lifecycle management activities.

In addition, process data from both the IVT reaction vessel 502 and the IVT hold vessel 504 is transmitted to the digital twin. The digital twin utilizes process data and soft-sensor data to model, predict, and optimize the unit operations of the system, including e.g., in vitro transcription and tangential flow filtration, as illustrated in FIG. 5A and FIG. 5B.

FIG. 5B shows integration of the IVT unit operation apparatus of FIG. 5A with a downstream TFF unit operation apparatus. The TFF unit apparatus includes a first tangential flow filtration unit, TFF filter unit 522 which receives process fluid 538 from the IVT hold vessel 504. Filtrate from the TFF filter unit 522 unit is sent to a TFF hold vessel 524 which is fluidly connected via a recirculation loop 528 to a UV spectrometer 526, optionally a variable pathlength UV spectrometer. The TFF filter unit 522 transmits a data stream 536 which includes process data measured by the TFF device such as pH, temperature, conductivity, pressure, and UV data.

In operation, the UV spectrometer measures the concentration of RNA and other CQAs. This data, indicated in the figure by dotted lines, is transmitted to the CQA controller 532 and the digital twin 202 (pictured in FIG. 5A).

In operation, the process described in FIG. 5A and FIG. 5B operates as follows. Process fluid from the IVT reaction vessel 502 recirculates between a first set of two PAT optical instruments, Raman 506 and UV 508. The first set of optical analytic instruments in combination with the CQA controller 514 monitor the progression of the IVT reaction and determine when the reaction has met a set of predetermined parameters. Upon reaching the set of predetermined parameters, the system initiates transfer of a volume of process fluid from the IVT reaction vessel 502 to the IVT hold vessel 504 via actuation of a pump and at least one valve (not shown) where it enters a recirculation loop with a second set of optical analytic instruments, Raman 510 and UV 512. The second set of optical analytic instruments in combination with the CQA controller 514 determine whether the volume in the IVT hold vessel 504 meets a predetermined set of CQAs. Where the CQAs are met, the system initiates transfer of the volume of process fluid from the IVT hold vessel 504 to a TFF filter unit 522 unit. The volume of process fluid flows through the TFF filter unit 522 unit and the filtrate is captured in a TFF hold vessel 524 where it enters a recirculation loop with a UV 526 optical analytic instrument. The UV 526 optical analytic instrument in combination with the CQA controller 514 determine whether the volume in the TFF hold vessel 524 meets a predetermined set of CQAs. Where the CQAs are met, the system initiates transfer of the volume of process fluid from the TFF hold vessel 524 to a downstream unit operation, such as a chromatography unit operation.

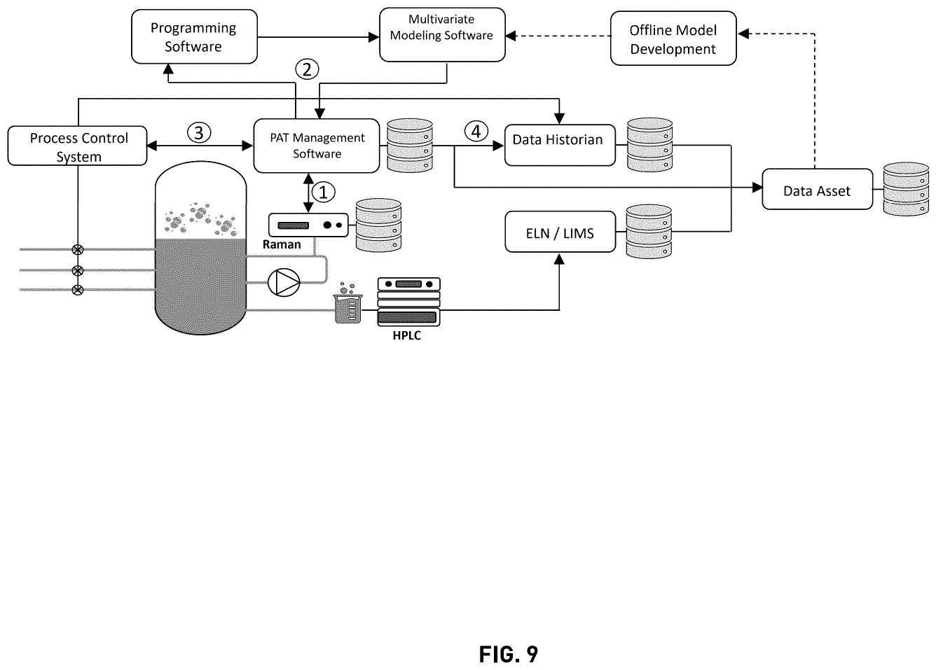

Fluid transfer in the system, e.g., between the IVT reaction vessel and the IVT hold vessel or between the IVT hold vessel and the TFF unit apparatus or between the TFF unit apparatus and the TFF hold vessel is accomplished by means of a series of pumps and valves which are actuated by the CQA controllers. Thus, in operation, for each unit apparatus, process data is sent to the CQA controller to verify that predetermined CQAs are satisfied. If the specifications are met, the CQA controller initiates transfer of the process fluid to the next downstream unit operation by a process that includes, for example, actuation of a pump and opening of a valve in the transfer line to move the process fluid from one vessel to the next. See FIG. 9 for a more detailed discussion of the flow of data in a unit operation.

In summary, the system provides for inline sampling of the process fluid via Raman and UV flow cells at the IVT reaction vessel and the IVT hold vessel, as well as inline sampling via a UV flow cell at the TFF hold vessel. Soft-sensors provide for real-time release testing at the IVT reaction vessel, the IVT hold vessel, and the TFF hold vessel, for example based on product RNA concentration, purity, and other CQAs pre-calibrated with off-line analytics. Finally, an optional digital twin utilizes process data and soft-sensor data to model, predict, and optimize the unit operations of the system, including e.g., in vitro transcription and tangential flow filtration.

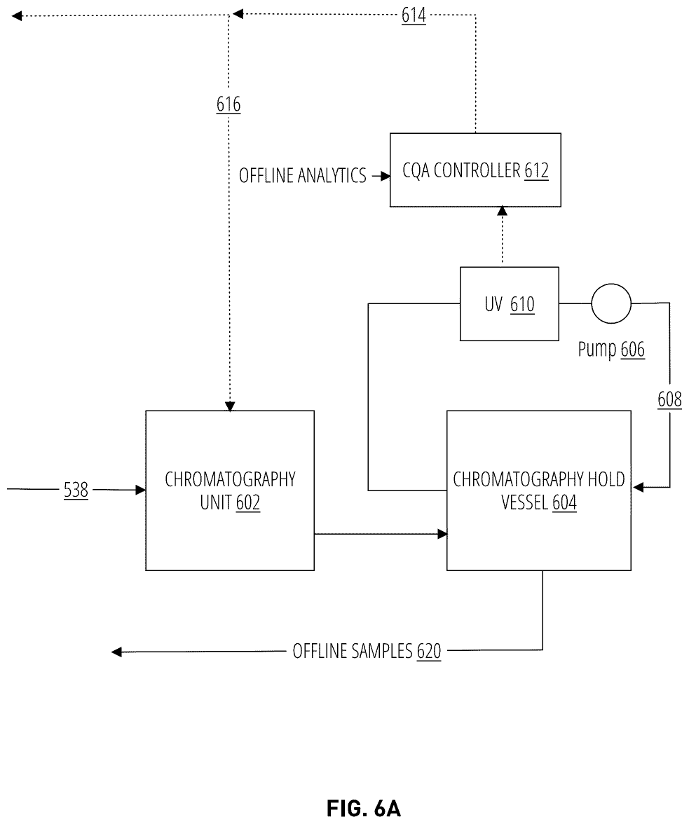

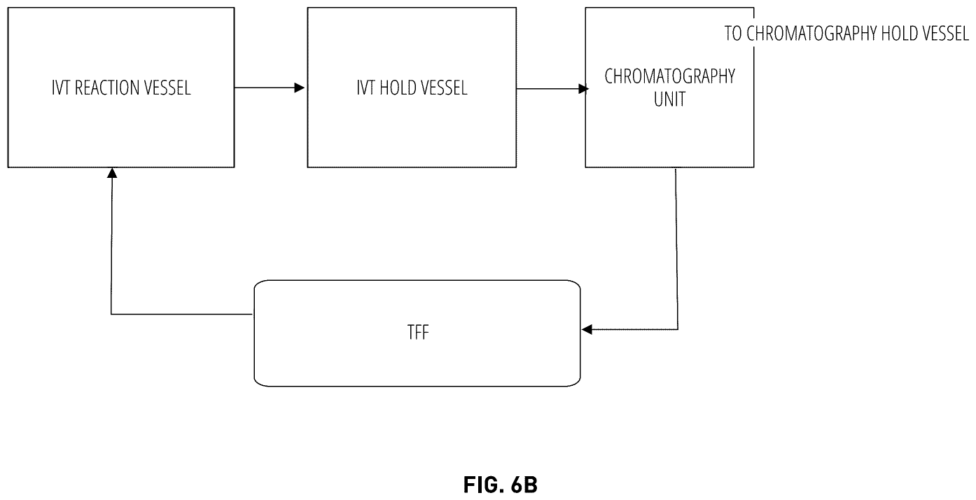

FIG. 6A shows integration of the IVT unit operation apparatus of FIG. 5A with a downstream chromatography unit 602 unit operation apparatus. In operation, the chromatography unit 602 apparatus receives process fluid 538 from the IVT hold vessel 504. Eluate from the chromatography unit 602 unit is sent to a chromatography hold vessel 604 which is fluidly connected via a recirculation loop 608 with a UV spectrometer 610, optionally a variable pathlength UV spectrometer. The UV spectrometer measures the concentration of RNA and optionally other CQAs. This UV process data, as well as additional process data obtained by sensors in the chromatography unit 602 itself such as pH, temperature, conductivity, pressure, and UV data, is transmitted to the CQA controller 612 and the digital twin 202 (pictured in FIG. 5A), indicated by dotted lines, 614 and 616.

FIG. 6B illustrates an aspect of the IVT and chromatography system of FIG. 6A where the system includes a TFF unit operation for recovery of certain reagents of the IVT reaction from the chromatography unit, which are recycled back into the IVT reaction vessel. Recycled reagents may include one or more of nucleotides, plasmid DNA template, capping enzyme, RNA polymerase, and pyrophosphatase enzyme.

In operation, the process described in FIG. 6A and FIG. 6B operates as follows. Process fluid from the IVT reaction vessel 502 recirculates between a first set of two PAT optical instruments, Raman 506 and UV 508. The first set of optical analytic instruments in combination with the CQA controller 514 monitor the progression of the IVT reaction and determine when the reaction has met a set of predetermined parameters. Upon reaching the set of predetermined parameters, the system initiates transfer of a volume of process fluid from the IVT reaction vessel 502 to the IVT hold vessel 504 via actuation of a pump (not shown) where it enters a recirculation loop with a second set of optical analytic instruments, Raman 510 and UV 512. The second set of optical analytic instruments in combination with the CQA controller 514 determine whether the volume in the IVT hold vessel 504 meets a predetermined set of CQAs. Where the CQAs are met, the system initiates transfer of the volume of process fluid from the IVT hold vessel 504 to a chromatography unit 602 unit. The volume of process fluid flows through the chromatography unit 602 unit and the eluate is captured in a chromatography hold vessel 604 where it enters a recirculation loop with a UV 610 optical analytic instrument. The UV 610 optical analytic instrument in combination with the CQA controller 514 determine whether the volume in the chromatography hold vessel 604 meets a predetermined set of CQAs. Where the CQAs are met, the system initiates transfer of the volume of process fluid from the chromatography hold vessel 604 to a downstream unit operation, such as a TFF unit operation. Where the system includes the optional TFF unit positioned between the chromatography unit apparatus and the IVT reaction vessel, reagents of the IVT reaction such as nucleotides, plasmid DNA template, capping enzyme, RNA polymerase, and/or pyrophosphatase enzyme are recovered via operation of the TFF unit and recycled back into the IVT reaction vessel.

Fluid transfer in the system, e.g., between the IVT reaction vessel and the IVT hold vessel or between the IVT hold vessel and the chromatography unit apparatus or between the chromatography unit apparatus and the chromatography hold vessel, or through the optional TFF unit located between the chromatography unit and the IVT reaction vessel, is accomplished by means of a series of pumps which are actuated by the CQA controllers.

In summary, the system provides for inline sampling of the process fluid via Raman and UV flow cells at the IVT reaction vessel and the IVT hold vessel, as well as inline sampling via a UV flow cell at the TFF hold vessel. Soft-sensors provide for real-time release testing at the IVT reaction vessel, the IVT hold vessel, and the TFF hold vessel, for example based on product RNA concentration, purity, and other CQAs pre-calibrated with off-line analytics. Finally, an optional digital twin utilizes process data and soft-sensor data to model, predict, and optimize the unit operations of the system, including e.g., in vitro transcription and tangential flow filtration.

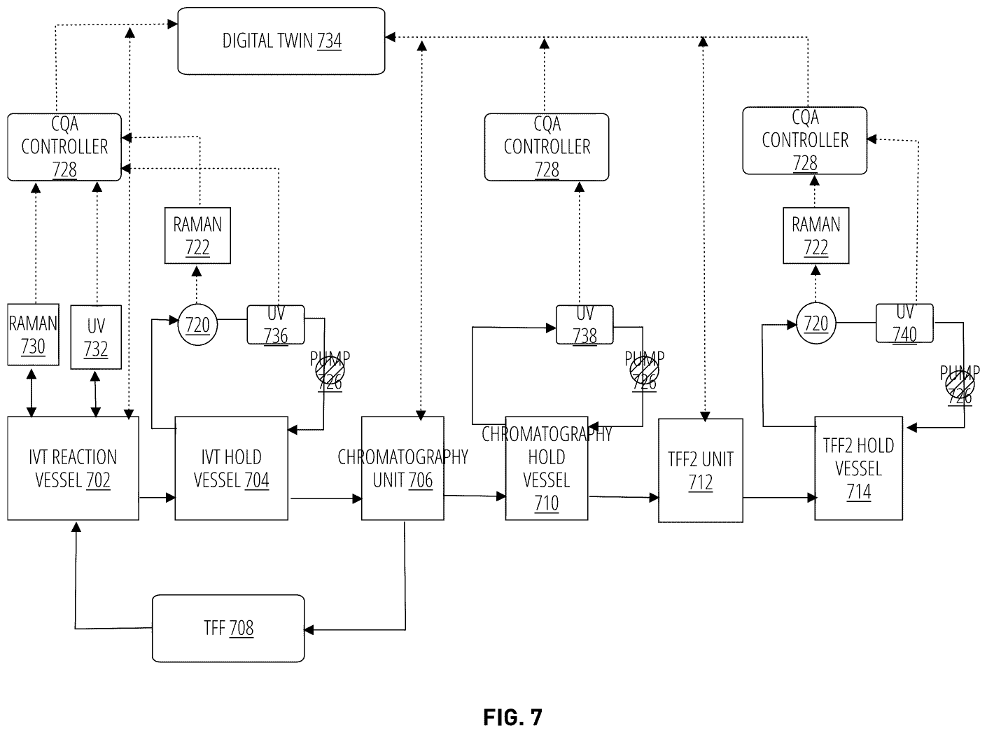

FIG. 7 illustrates an IVT system in accordance with one embodiment. The system includes an IVT unit apparatus, a chromatography unit apparatus, and a TFF unit apparatus. Also pictured is and optional TFF unit fluidly connected between the chromatography unit and the IVT reaction vessel for recycling of one or more reagents of the IVT reaction such as nucleotides, plasmid DNA template, capping enzyme, RNA polymerase, and pyrophosphatase enzyme.

In operation, process fluid from the IVT reaction vessel 702 recirculates between a first set of two optical analytic optical instruments, Raman 730 and UV 732. The first set of optical analytic instruments in combination with the CQA controller 728 monitor the progression of the IVT reaction and determine when the reaction has met a set of predetermined parameters. Upon reaching the set of predetermined parameters, the system initiates transfer of a volume of process fluid from the IVT reaction vessel 702 to the IVT hold vessel 704 via actuation of a pump (not shown) where it enters a recirculation loop with a second set of optical analytic instruments, Raman 722 and UV 716. The second set of optical analytic instruments in combination with the CQA controller 728 determine whether the volume in the IVT hold vessel 704 meets a predetermined set of CQAs. Where the CQAs are met, the system initiates transfer of the volume of process fluid from the IVT hold vessel 704 to the chromatography unit 706. The volume of process fluid flows through the chromatography unit 706 and the eluate is captured in a chromatography hold vessel 710 where it enters a recirculation loop with a UV 716 optical analytic instrument. The UV 716 optical analytic instrument in combination with the CQA controller 728 determine whether the volume in the chromatography hold vessel 710 meets a predetermined set of CQAs. Where the CQAs are met, the system initiates transfer of the volume of process fluid from the chromatography hold vessel 710 to the TFF2 unit 712. The process fluid flows through the filtration unit and the filtrate is captured in a TFF2 hold vessel 714 where it enters a recirculation loop with a third set of optical analytic instruments, Raman 722 and UV 716. Where the system includes the optional TFF unit positioned between the chromatography unit apparatus and the IVT reaction vessel, one or more reagents of the IVT reaction such as nucleotides, plasmid DNA template, capping enzyme, RNA polymerase, and/or pyrophosphatase enzyme are recovered via operation of the TFF unit and recycled back into the IVT reaction vessel.

Accordingly, the system provides for inline sampling of the process fluid via Raman and UV flow cells at the IVT reaction vessel 702, the IVT hold vessel 704, and the TFF2 hold vessel 714, as well as inline sampling via a UV flow cell at the chromatography hold vessel 710. Soft-sensors provide for real-time release testing at the IVT reaction vessel 702, the IVT hold vessel 704, the TFF2 hold vessel 714, and the chromatography hold vessel 710, for example based on product RNA concentration, purity, and other CQAs pre-calibrated with off-line analytics.

In aspects, the system may also include a digital twin 734 which utilizes process data and soft-sensor data to model, predict, and optimize the unit operations of in vitro transcription, chromatography, and tangential flow filtration.

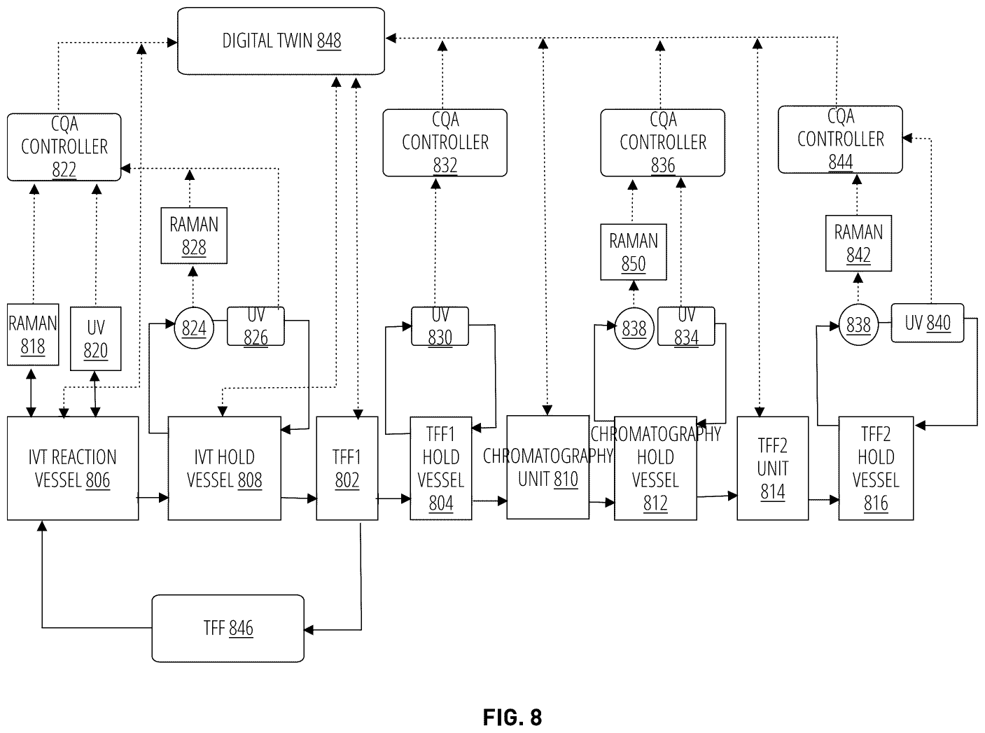

FIG. 8 illustrates an IVT system in accordance with an embodiment similar to that described above in connection with FIG. 7 except the system also includes a TFF unit apparatus between the IVT hold vessel and the chromatography unit apparatus. In operation, the system performs in a manner analogous to that described for the system depicted in FIG. 7. Also shown in FIG. 8 is the optional TFF loop between the chromatography unit and the IVT reaction vessel for recycling one or more reagents of the IVT reaction back into the IVT reaction vessel. The one or more reagents may include nucleotides, plasmid DNA template, capping enzyme, RNA polymerase, and pyrophosphatase enzyme.

The figure illustrates points in the process where real-time monitoring using Raman spectroscopy may be utilized. As illustrated, Raman spectroscopy may be utilized (1) following the IVT reaction, for example at the IVT hold vessel, to measure the concentration of RNA product, the concentration of reagents of the IVT reaction, which may also be referred to as raw materials, and impurities; (2) following chromatography, for example at the chromatography hold vessel, to measure the concentration of RNA product and confirm removal of impurities; and (3) following a post-chromatography TFF to measure the concentration of RNA product and confirm removal of impurities prior to lipid nanoparticle formulation of the final drug product.

FIG. 9 schematically shows the flow of data in a single unit operation, using the IVT operation for purposes of exemplification. As shown, multiple platforms are integrated to achieve fully automated monitoring. Represented in the figure are discrete elements of the CQA controller including PAT management software, multivariate modeling software, and a process control system (PCS) which may be in the form of a Supervisory Control and Data Acquisition (SCADA) system. In operation, data gathered from a Raman flow cell sampling point is sent to the PAT management software (1). The data represented in (1) include spectral data and measurement information from the instrument. The (1) arrow is represented as double-headed because the PAT management software also sends commands to the instrument, e.g., stop/start commands. In (2), the PAT management software sends data, including spectral data and orchestration information, to the multivariate modeling software (MVM). In turn, the MVM produces real-time predictions and diagnostics and returns data to the PAT management software. Model predictions and diagnostics are transmitted (3) from the PAT management software to the PCS and the PCS in turn transmits start/stop commands to the PAT management software. The PAT management software also transmits measurement information, model predictions, and diagnostics to a data historian for recording.

Also illustrated are offline sample collections which are acquired and analyzed by offline instruments such as HPLC and stored in an electronic lab notebook (ELN) and/or a laboratory inventory management system (LIMS). Data from offline and online instruments is aligned using data engineering tools to build data assets for more efficient model development and data analysis.

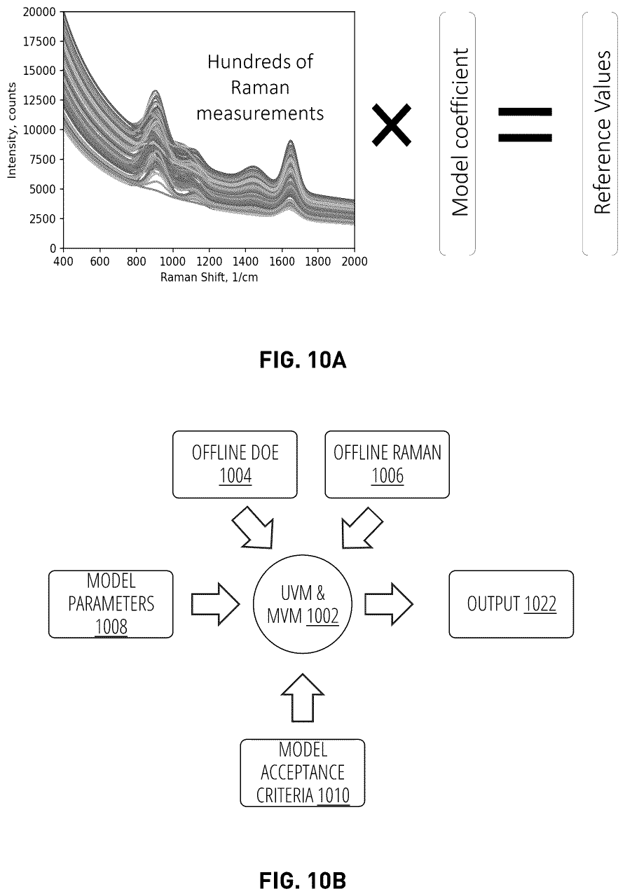

FIG. 10A schematically illustrates how data driven models are calibrated. Illustrated are hundreds of Raman spectra, represented as overlapping lines on the line graph, which are utilized by machine learning models to extract information, in this case to predict RNA product concentration based on Raman spectra. A large number of spectra may be used to achieve an acceptable statistical significance. The figure illustrates the mapping of multiple two-dimensional spectra into reference values obtained by offline analytics. The result is model coefficients which are used by the CQA controller to predict RNA concentration in real-time based on inline Raman measurements.

Multivariate models are particularly well-adapted to the task of extracting information from Raman spectra, at least in part because Raman spectra comprise multivariate data. In one implementation, a computer script written in a suitable language, such as Python, may be utilized to automate the process of probing the parameter space and identifying optimal parameters to determine modeling parameters and meet model validation criteria.

The graphic in FIG. 10B illustrates the automation of model development and optimization using a suitable script, such as a Python script. In some implementations, the offline DOE 1004 studies may include adding variations for raw materials and instruments and identifying fingerprint regions. The offline Raman 1006 data may include capturing process variations such as variations in process runs, constructs, raw materials, instruments, and sampling. Model parameters 1008 include preprocessing and partial least squares (PLS) regression parameters. Model acceptance criteria 1010 include r-squared (R2), root mean square error (RMSE), and validation criteria. The outputs 1022 of the automated model optimization include media components that can be modeled, specificity and univariate analyses, optimum parameters for multivariate modeling, statistical significance of the models, and generic model evaluation.

Taking the IVT process as an example, the relationship between the input variables and key outputs may be evaluated using a structured approach as follows:
1.Determination of process parameters for real-time monitoring.
2.Offline DOE experiments to determine Raman signature of raw materials and product.
3.Determination of measurement setup and strategy.
4.Collecting data from multiple runs and initial modeling.
5.Univariate analysis for qualitative trending.
6.Multivariate analysis for quantitative trending.

Raw materials, enzymes (in the form of a reaction mixture), and treatment agents are added sequentially to the IVT reaction vessel. Results will be a mixture of RNA product, remaining raw materials, enzymes and reaction byproducts.

The Raman spectra is obtained from the IVT reaction vessel during the transcription reaction to capture changes in the mixture due to the formation of RNA, byproducts such as phosphate groups, and consumption of the starting materials. As more RNA and byproduct are formed, certain Raman peaks increase in intensity while other peaks decrease in intensity due to the consumption of the starting materials. A univariate analysis is performed by normalizing the Raman spectra and trending the peaks with time. The trending plots reveal the pace and endpoint of the transcription reaction. A multivariate analysis is also performed by calculating principal components of the Raman spectra. The loading vectors of the principal component analysis (PCA) reveal critical spectral regions corresponding to the largest variations in intensity. These regions are correlated to the Raman signatures of the RNA, phosphate groups, and the starting materials.

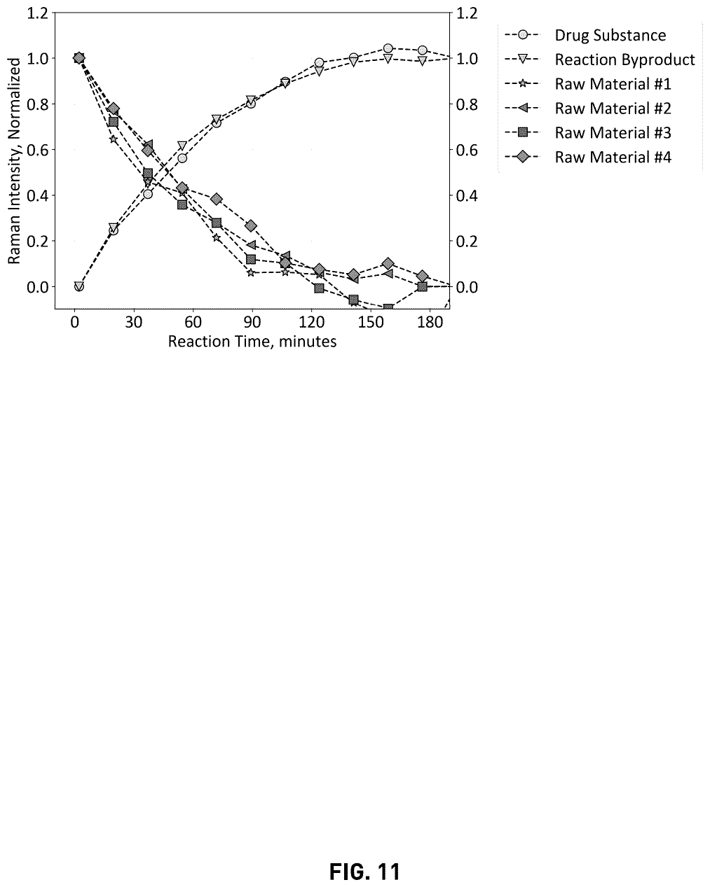

FIG. 11 illustrates real-time monitoring for in vitro transcription using a univariate analysis. The line graph shows normalized Raman intensity versus transcription time in an IVT unit operation for the Raman peaks of mRNA, reaction byproducts, and starting raw materials. The Raman signatures modelled include (1) drug substance and phosphate byproducts, which increase and plateau after 150 minutes; and (2) raw materials (NTPs), which decrease over time indicating that the reaction might stop with the full consumption of one of the raw materials. The univariate analysis also provides a qualitative trend useful to understand the reaction pace and endpoint.

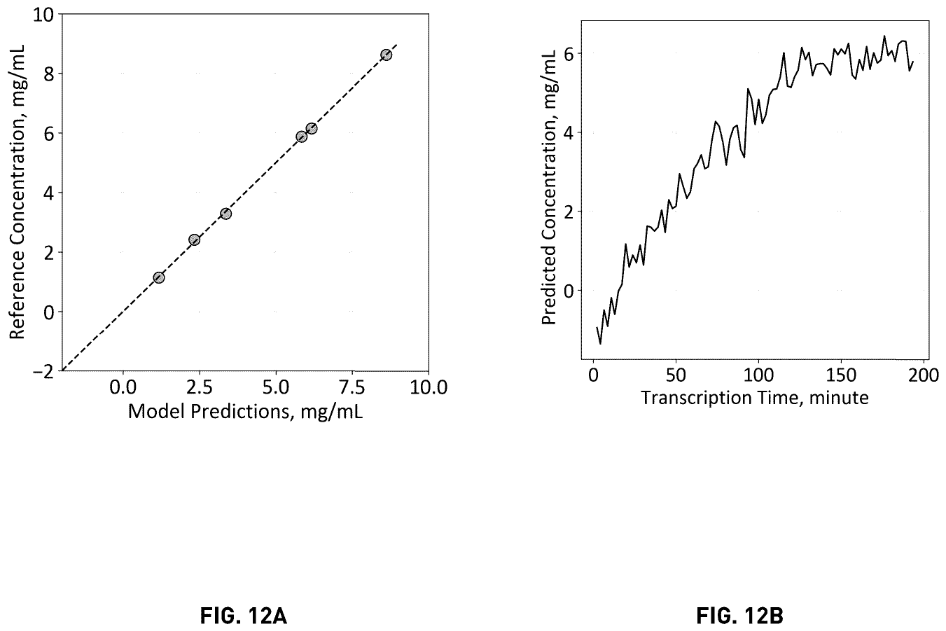

FIG. 12A illustrates real-time monitoring for in vitro transcription using a multivariate analysis. The line graph shows a partial least squares (PLS) regression between the model prediction for RNA concentration (mg/ml) and the reference RNA concentration (mg/ml) obtained via offline measurement. A regression R² of above 0.99 and low root mean-squared error (RMSE) were achieved (RMSE=0.05 mg/ml) for the model calibration.

FIG. 12B illustrates how the PLS model was used to predict an independent run mRNA concentration based on transcription time (minutes). The PLS model prediction for an independent run was 6.0 mg/mL compared to the offline measurement of 6.8 mg/mL that was measured using Thermo Fisher's NanoDrop™ UV-Vis Spectrophotometer. Under the modeled conditions, RNA increases with time and reaches a plateau in about 120 minutes. The final concentration confirmed with the offline measurement with around 12% error.

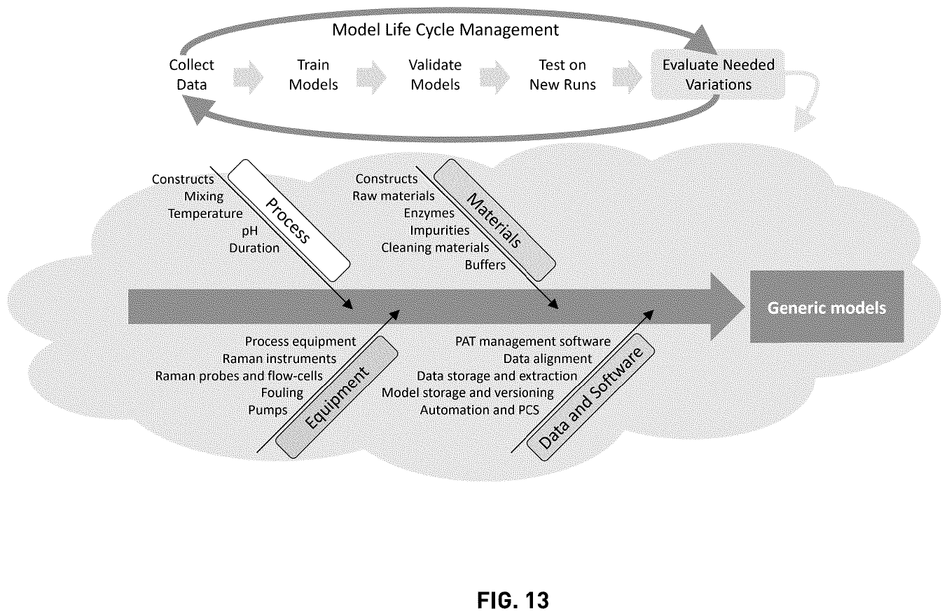

FIG. 13 13 illustrates multivariate model life cycle management and development of generic models. Multivariate models are calibrated by collecting data from online and offline analytical instruments and validated by data sets collected from runs that are not included in the calibration data set. Once the validation criteria are met, the multivariate models will be tested on future runs to monitor process in real-time as well as to evaluate model robustness and re-calibration. To develop generic models, possible variations should be included in the model calibration. The variation sources are from process (mixing, temperature, pH, and duration), materials (RNA constructs, raw materials, enzymes, impurities, cleaning agents, and buffers), equipment (process unit operations, analytical instruments, probes, flow cells, pumps, and tubes), and data.

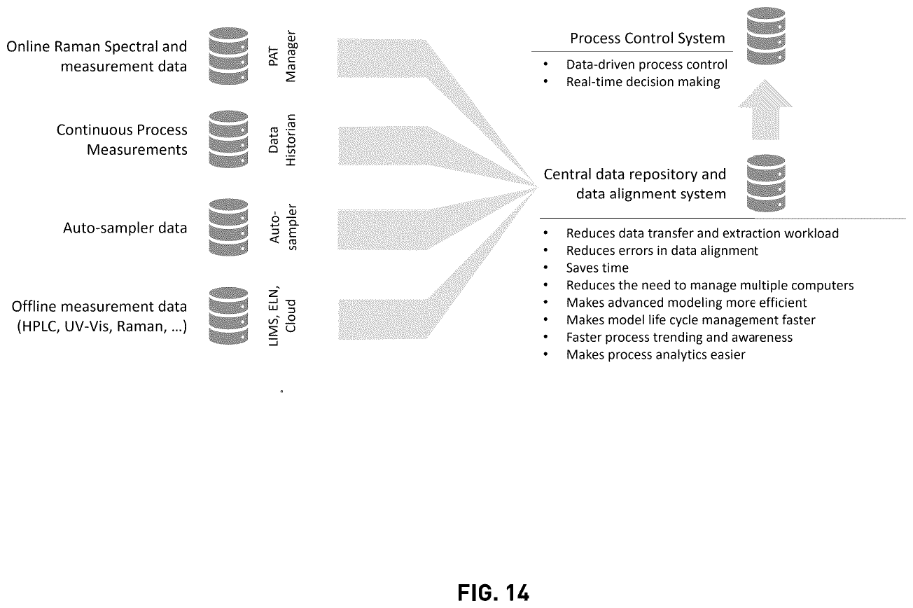

FIG. 14 is a schematic illustrating the importance of data flow and management. The central data repository and data alignment management system will allow for reduction in workload of data transfer, extraction, and errors in alignment. This allows for time savings, agile model lifecycle management (model development and implementation), and more efficient process trending and control.

### EXAMPLE 1: MULTIVARIATE PLS MODELLING WITH IN-LINE RAMAN TO PREDICT MRNA CONCENTRATION IN IVT REACTION

Multivariate PLS modelling was used with in-line Raman Spectroscopy to predict mRNA concentration in IVT. The process included the following runs: Run CP1-Fluc-004, CP1-Fluc-005, CP1-Fluc-006, CP1-Fluc-008, CP1-Covid-010, CP1-Covid-012, CPC-Fluc-001, and CPC-Fluc-002. These runs were used to train the PLS model. A total of 52 samples were run.

Run CP1-Fluc-007, CP1-Covid-009, and CPC-Fluc-003 were used to validate the PLS model. A total of 22 samples from independent runs were used.

Three different reaction scales (10 ml, 25 ml, 250 ml) were included in the model training. Therefore, reaction scale variation is incorporated into the PLS model.

Two different Raman instruments and flow cells were included in the model training. Therefore, instrument variation is incorporated into the PLS model.

Two different mRNA constructs (Fluc and Covid) were included in the model training. Therefore, construct variation is incorporated into the PLS model. Therefore, the trained PLS model is agnostic of scale, Raman instrument, and mRNA construct.

TABLE 1
Sample list from CP1 and CPC process used for
PLS model development for mRNA monitoring

| | | | | | Off-line mRNA | |
|---|---|---|---|---|---|---|
| | | Scale, | | Sample Time, | Concentration, | |
| Number | Run ID | ml | Construct | minutes | mg/ml | Used for |
| 1 | CP1-Fluc-004 | 250 | Fluc | 0 | 1.14 | Training |
| 2 | CP1-Fluc-004 | 250 | Fluc | 36 | 2.41 | Training |
| 3 | CP1-Fluc-004 | 250 | Fluc | 72 | 3.29 | Training |
| 4 | CP1-Fluc-004 | 250 | Fluc | 109 | 5.87 | Training |
| 5 | CP1-Fluc-004 | 250 | Fluc | 145 | 6.15 | Training |
| 6 | CP1-Fluc-005 | 250 | Fluc | 0 | 0.93 | Training |
| 7 | CP1-Fluc-005 | 250 | Fluc | 36 | 2.56 | Training |
| 8 | CP1-Fluc-005 | 250 | Fluc | 72 | 4.26 | Training |
| 9 | CP1-Fluc-005 | 250 | Fluc | 109 | 6.45 | Training |
| 10 | CP1-Fluc-005 | 250 | Fluc | 145 | 6.77 | Training |
| 11 | CP1-Fluc-006 | 250 | Fluc | 0 | 1.31 | Training |
| 12 | CP1-Fluc-006 | 250 | Fluc | 36 | 3.60 | Training |
| 13 | CP1-Fluc-006 | 250 | Fluc | 72 | 4.30 | Training |
| 14 | CP1-Fluc-006 | 250 | Fluc | 109 | 6.60 | Training |
| 15 | CP1-Fluc-006 | 250 | Fluc | 145 | 6.41 | Training |
| 16 | CP1-Fluc-007 | 250 | Fluc | 0 | 0.78 | Validation |
| 17 | CP1-Fluc-007 | 250 | Fluc | 36 | 4.14 | Validation |
| 18 | CP1-Fluc-007 | 250 | Fluc | 72 | 5.93 | Validation |
| 19 | CP1-Fluc-007 | 250 | Fluc | 109 | 7.16 | Validation |
| 20 | CP1-Fluc-007 | 250 | Fluc | 145 | 7.58 | Validation |
| 21 | CP1-Fluc-008 | 250 | Fluc | 0 | 0.24 | Training |
| 22 | CP1-Fluc-008 | 250 | Fluc | 36 | 3.11 | Training |
| 23 | CP1-Fluc-008 | 250 | Fluc | 72 | 5.88 | Training |
| 24 | CP1-Fluc-008 | 250 | Fluc | 109 | 8.07 | Training |
| 25 | CP1-Fluc-008 | 250 | Fluc | 145 | 8.21 | Training |
| 26 | CP1-Covid-009 | 250 | Covid | 0 | 0.17 | Validation |
| 27 | CP1-Covid-009 | 250 | Covid | 36 | 0.92 | Validation |
| 28 | CP1-Covid-009 | 250 | Covid | 72 | 1.82 | Validation |
| 29 | CP1-Covid-009 | 250 | Covid | 109 | 3.12 | Validation |
| 30 | CP1-Covid-009 | 250 | Covid | 145 | 5.37 | Validation |
| 31 | CP1-Covid-010 | 250 | Covid | 0 | 0.13 | Training |
| 32 | CP1-Covid-010 | 250 | Covid | 36 | 0.61 | Training |
| 33 | CP1-Covid-010 | 250 | Covid | 72 | 1.59 | Training |
| 34 | CP1-Covid-010 | 250 | Covid | 109 | 2.77 | Training |
| 35 | CP1-Covid-010 | 250 | Covid | 145 | 4.17 | Training |
| 36 | CP1-Covid-012 | 250 | Covid | 0 | 0.05 | Training |
| 37 | CP1-Covid-012 | 250 | Covid | 36 | 1.06 | Training |
| 38 | CP1-Covid-012 | 250 | Covid | 72 | 1.27 | Training |
| 39 | CP1-Covid-012 | 250 | Covid | 109 | 2.3 | Training |
| 40 | CP1-Covid-012 | 250 | Covid | 145 | 3.66 | Training |
| 41 | CP1-Covid-012 | 250 | Covid | 182 | 5.03 | Training |
| 42 | CP1-Covid-012 | 250 | Covid | 216 | 6.84 | Training |
| 43 | CP1-Covid-012 | 250 | Covid | 252 | 7.66 | Training |
| 44 | CP1-Covid-012 | 250 | Covid | 271 | 8.75 | Training |
| 45 | CPC-Fluc-001 | 10 | Fluc | 30 | 3.00 | Training |
| 46 | CPC-Fluc-001 | 10 | Fluc | 60 | 5.10 | Training |
| 47 | CPC-Fluc-001 | 10 | Fluc | 90 | 6.10 | Training |
| 48 | CPC-Fluc-001 | 10 | Fluc | 120 | 6.20 | Training |
| 49 | CPC-Fluc-001 | 10 | Fluc | 150 | 6.80 | Training |
| 50 | CPC-Fluc-001 | 10 | Fluc | 180 | 6.60 | Training |
| 51 | CPC-Fluc-002 | 25 | Fluc | 15 | 1.49 | Training |
| 52 | CPC-Fluc-002 | 25 | Fluc | 30 | 2.13 | Training |
| 53 | CPC-Fluc-002 | 25 | Fluc | 45 | 2.84 | Training |
| 54 | CPC-Fluc-002 | 25 | Fluc | 60 | 4.75 | Training |
| 55 | CPC-Fluc-002 | 25 | Fluc | 75 | 4.81 | Training |
| 56 | CPC-Fluc-002 | 25 | Fluc | 90 | 6.07 | Training |
| 57 | CPC-Fluc-002 | 25 | Fluc | 105 | 4.43 | Training |
| 58 | CPC-Fluc-002 | 25 | Fluc | 120 | 4.86 | Training |
| 59 | CPC-Fluc-002 | 25 | Fluc | 135 | 5.37 | Training |
| 60 | CPC-Fluc-002 | 25 | Fluc | 150 | 5.01 | Training |
| 61 | CPC-Fluc-002 | 25 | Fluc | 165 | 5.05 | Training |
| 62 | CPC-Fluc-002 | 25 | Fluc | 180 | 4.88 | Training |
| 63 | CPC-Fluc-003 | 25 | Fluc | 15 | 1.82 | Validation |
| 64 | CPC-Fluc-003 | 25 | Fluc | 30 | 2.61 | Validation |
| 65 | CPC-Fluc-003 | 25 | Fluc | 45 | 3.67 | Validation |
| 66 | CPC-Fluc-003 | 25 | Fluc | 60 | 4.72 | Validation |
| 67 | CPC-Fluc-003 | 25 | Fluc | 75 | 5.09 | Validation |
| 68 | CPC-Fluc-003 | 25 | Fluc | 90 | 5.46 | Validation |
| 69 | CPC-Fluc-003 | 25 | Fluc | 105 | 8.35 | Validation |
| 70 | CPC-Fluc-003 | 25 | Fluc | 120 | 7.52 | Validation |
| 71 | CPC-Fluc-003 | 25 | Fluc | 135 | 6.67 | Validation |
| 72 | CPC-Fluc-003 | 25 | Fluc | 150 | 9.18 | Validation |
| 73 | CPC-Fluc-003 | 25 | Fluc | 165 | 6.97 | Validation |
| 74 | CPC-Fluc-003 | 25 | Fluc | 180 | 5.18 | Validation |

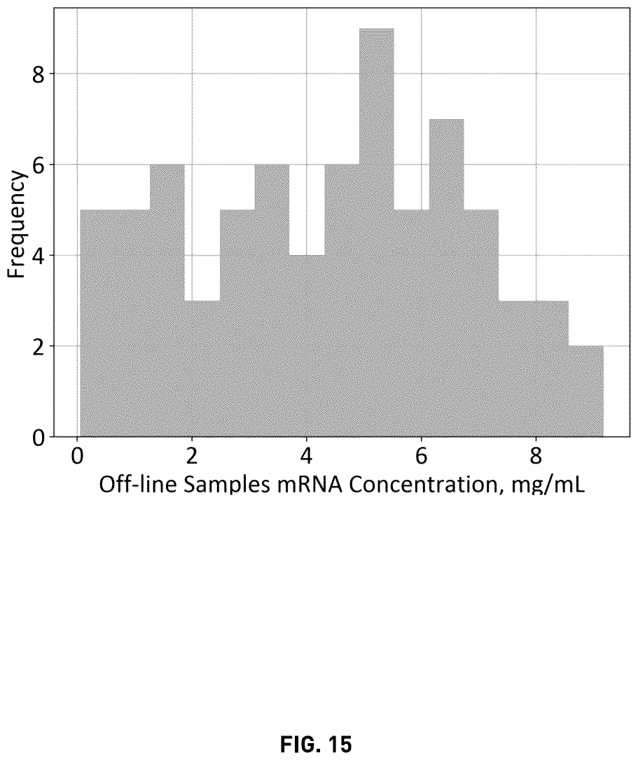

FIG. 15 is a graph illustrating a distribution of samples according to the mRNA concentration of the samples. This histogram illustrates the samples used in model development. The histogram represents every mRNA concentration in the range of 0.05 mg/ml to 9.18 mg/ml.

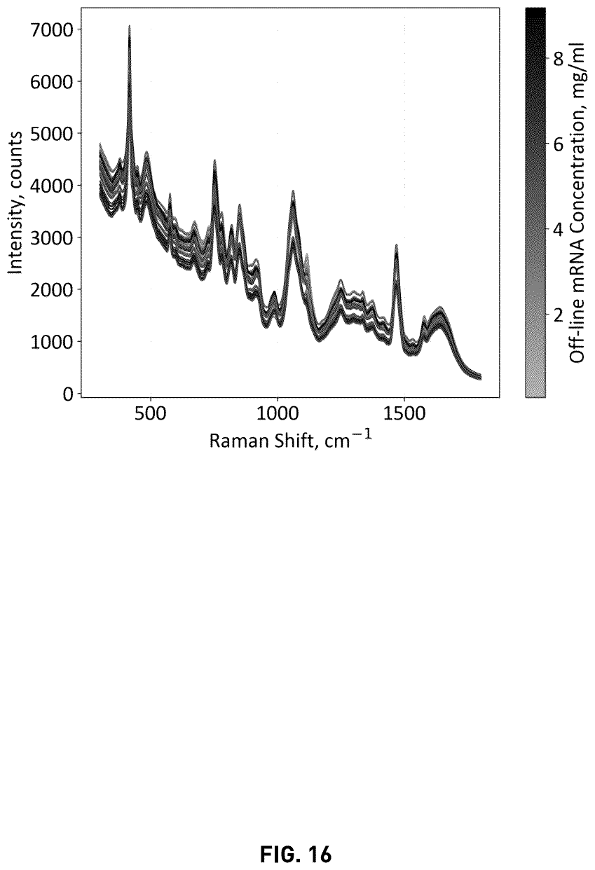

FIG. 16 is a line graph of raw spectral data acquired from IVT runs using a in-line Raman flow cell. The intensity of the raw spectral data does not correlate to mRNA concentration and therefore spectral data needs to be pre-processed for model training.

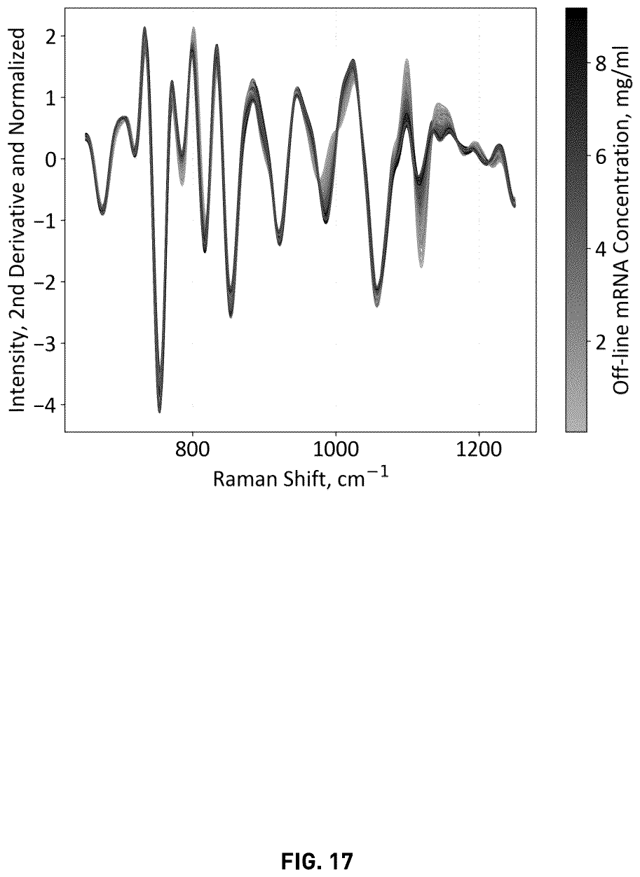

FIG. 17 is a line graph of a normalized 2^{nd} derivative of spectral data acquired from IVT runs using in-line Raman flow cell. After taking 2^{nd} derivative and normalizing the spectral data, a correlation appears between pre-processed intensity and mRNA concentration.

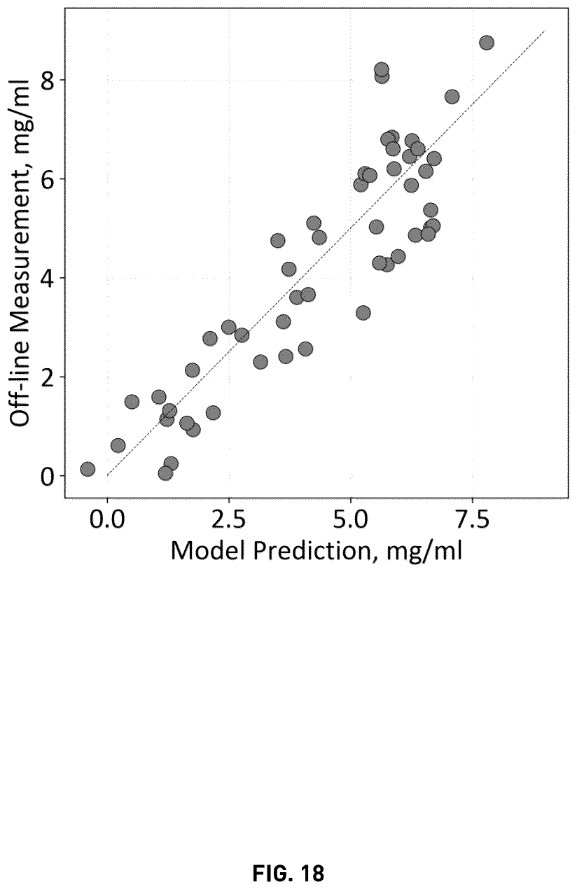

FIG. 18 is a regression plot for model training illustrating that the model predictions correlate to the off-line measurements. RMSE of cross-validated dataset=1.04 mg/ml; RMSE of training dataset=0.82 mg/ml; R²=0.87; Q²=0.76.

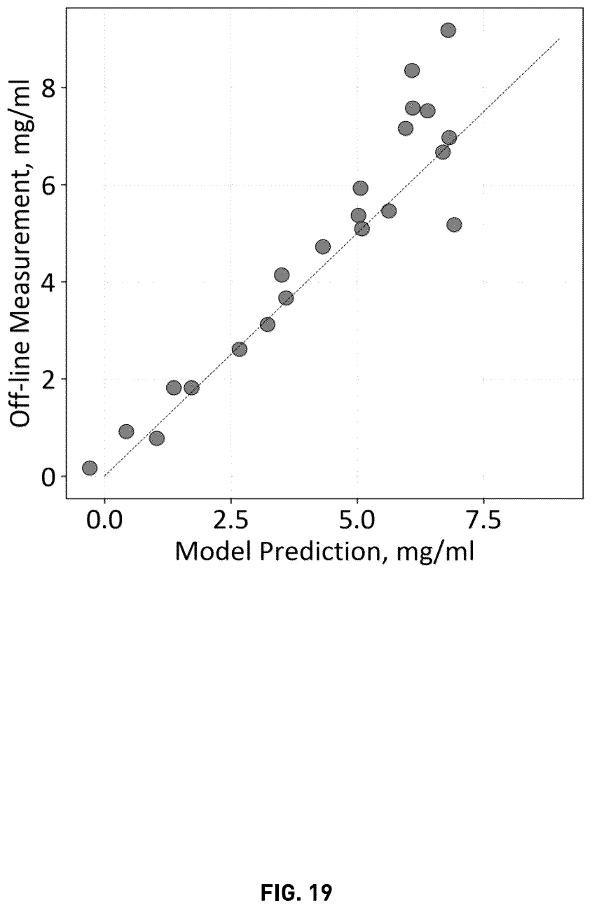

FIG. 19 is a regression plot for model validation illustrating that the model prediction correlates to the off-line concentration. RMSE of validation dataset=0.98 mg/ml; R² of validation dataset=0.81.

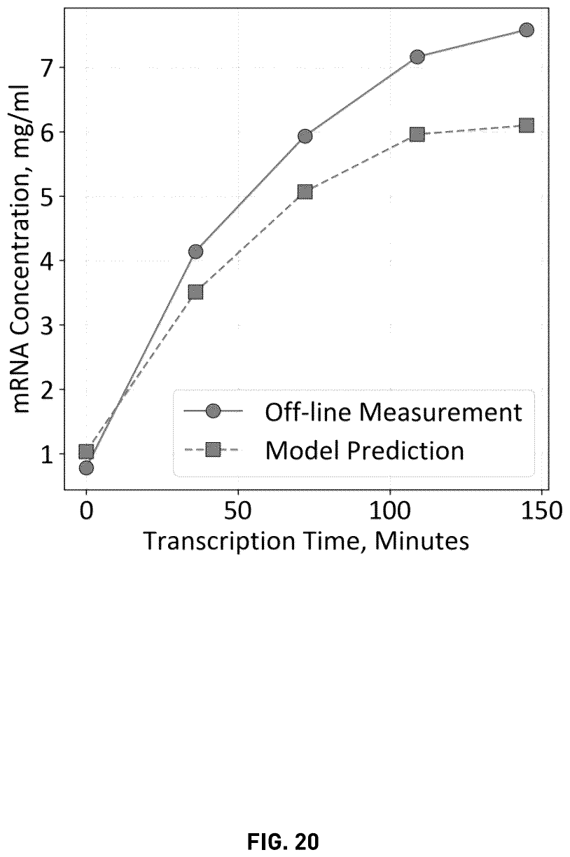

FIG. 20 is a line graph illustrating model predictions for the validation dataset run CP1-Fluc-007 compared to mRNA concentration measured off-line.

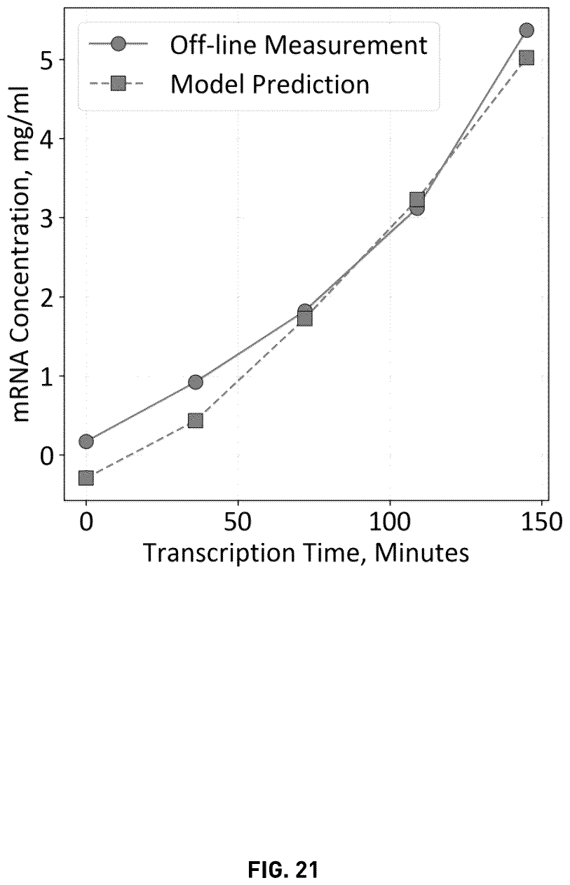

FIG. 21 is a line graph illustrating model predictions for the validation dataset run CP1-Covid-009 compared to mRNA concentration measured off-line.

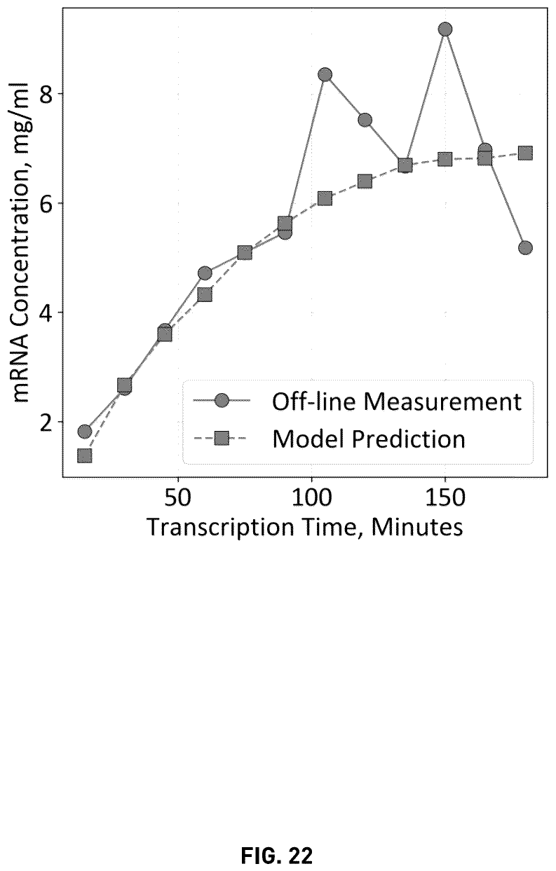

FIG. 22 is a line graph illustrating model predictions for the validation dataset run CP1-Fluc-003 compared to mRNA concentration measured off-line.

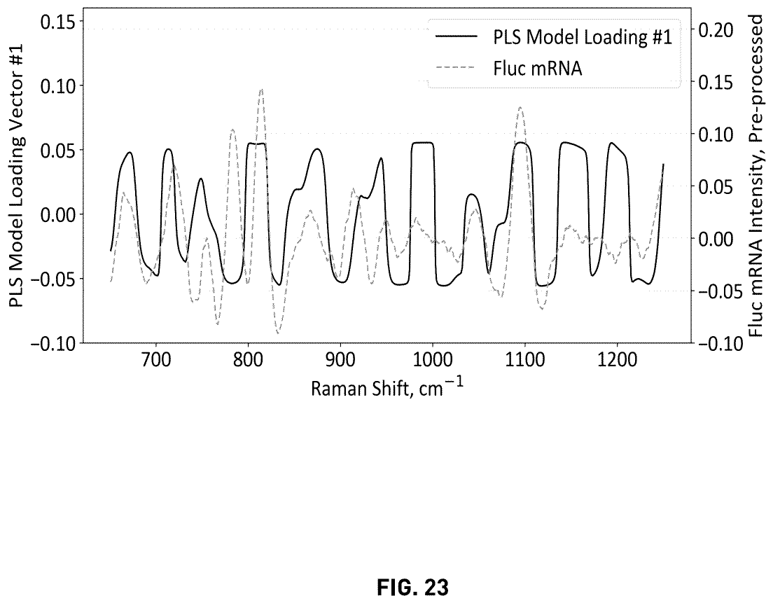

FIG. 23 is a line graph of PLS model loading vector #1 vs mRNA Raman spectrum illustrating specificity of the model to mRNA.

The PLS model loading vector #1 aligns with mRNA Raman spectrum around major mRNA peaks. These peaks occur around 800 cm⁻¹ and 1100 cm⁻¹ Raman shifts.

### EXAMPLE 2: UNIVARIATE ANALYSIS WITH IN-LINE RAMAN TO MONITOR IVT REACTION

In this example a univariate analysis with in-line Raman spectroscopy was used to monitor the IVT reaction. Raman spectral data was gathered every two minutes during the IVT reaction. Raman spectra was normalized using a standard normal variate (SNV) technique. Normalized spectra were subtracted by the starting material spectra to visualize growth and consumption in Raman peaks.

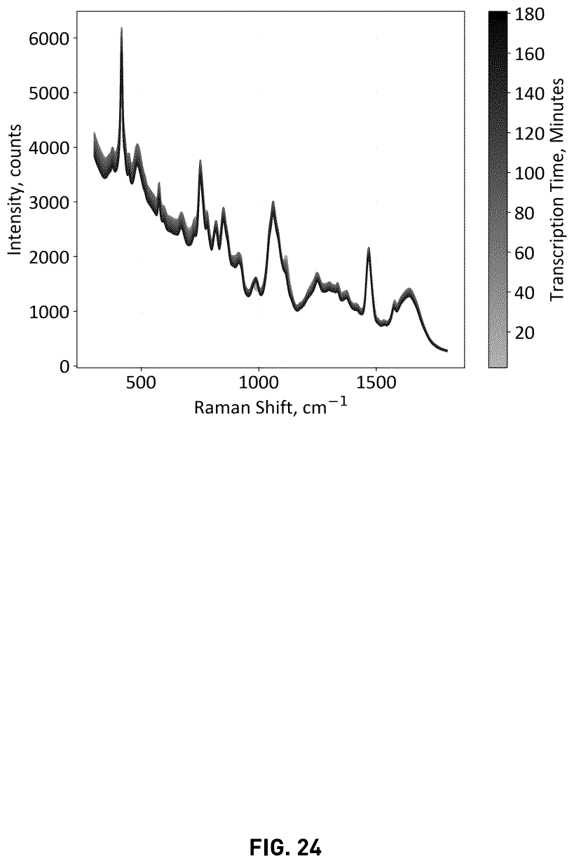

FIG. 24 is a line graph of raw spectral data acquired from IVT during CPC-Fluc-003 run using an in-line Raman flow cell. The greyscale changes in the data highlight the changes in the Raman intensity as a function of the transcription time.

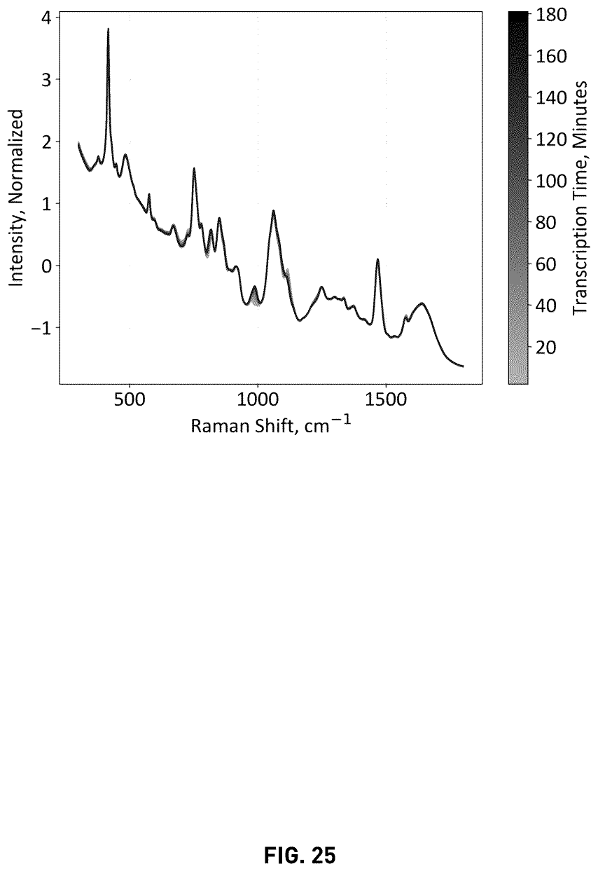

FIG. 25 is a line graph of normalized spectral data acquired from IVT during CPC-Fluc-003 run an using in-line Raman flow cell. The greyscale changes in the data highlight the changes in the Raman intensity as a function of the transcription time.

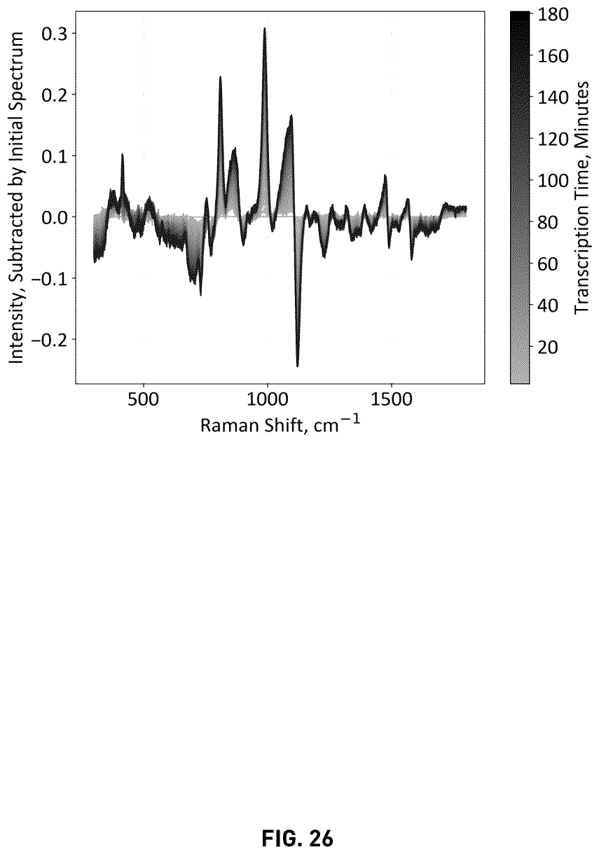

FIG. 26 is a line graph of normalized spectral data acquired from IVT during CPC-Fluc-003 run using in-line Raman flow cell. The greyscale changes in the data highlight the changes in the Raman intensity as a function of the transcription time. By subtracting initial spectrum from all spectra, changes in the Raman spectra due to mRNA transcription becomes clearer.

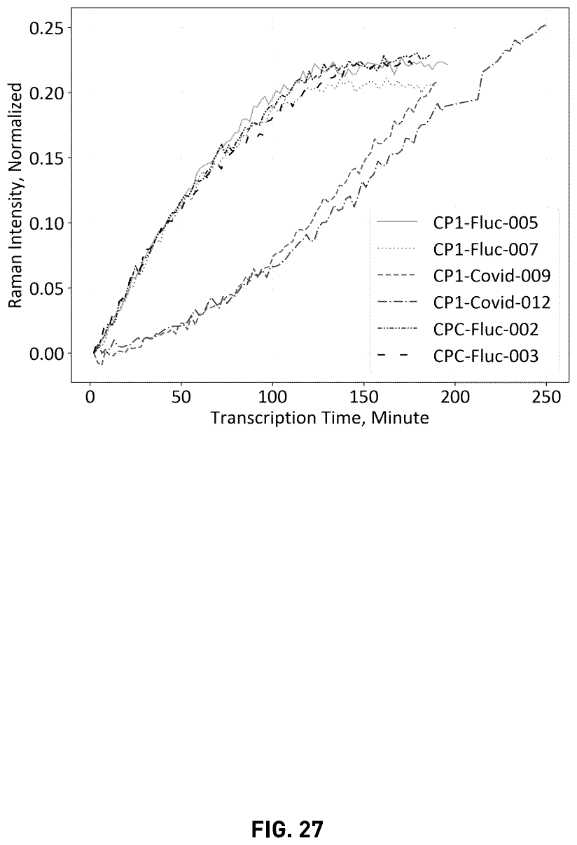

FIG. 27 is a graph illustrating Raman intensity at 812 cm⁻¹ as a function of transcription time. The Raman peak at 812 cm⁻¹ corresponds to the phosphodiester bond connecting nucleotides together in mRNA. This plot shows that the mRNA transcription pace depends on the mRNA construct. This plot also shows that mRNA transcription plateaus at around the 120^{th} minute regardless of IVT scale (CP1 and CPC IVT conducted at 250 ml and 25 ml reactors respectively).

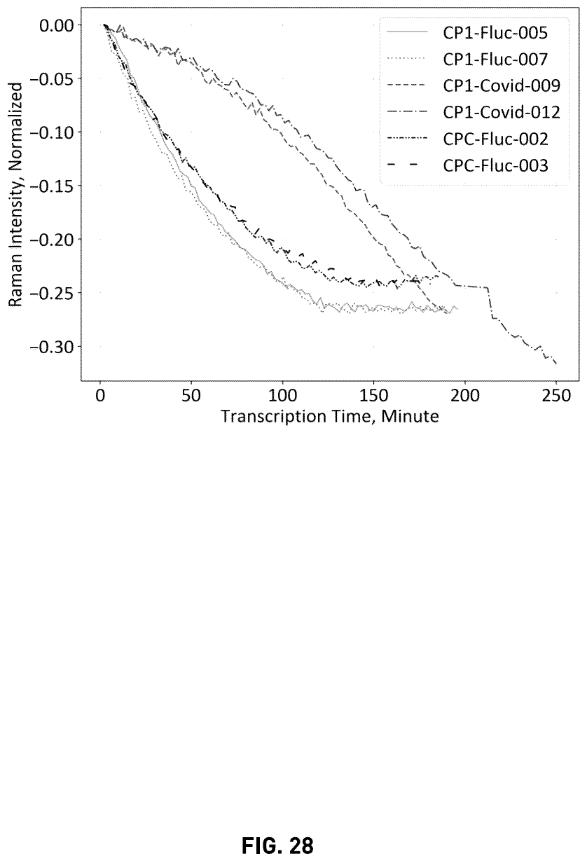

FIG. 28 is a graph illustrating Raman intensity at 1122 cm⁻¹ as a function of transcription time. The Raman peak at 1122 cm⁻¹ corresponds to the triphosphate tale of NTPs that break down during mRNA transcription reaction and therefore corresponding Raman intensity drops.

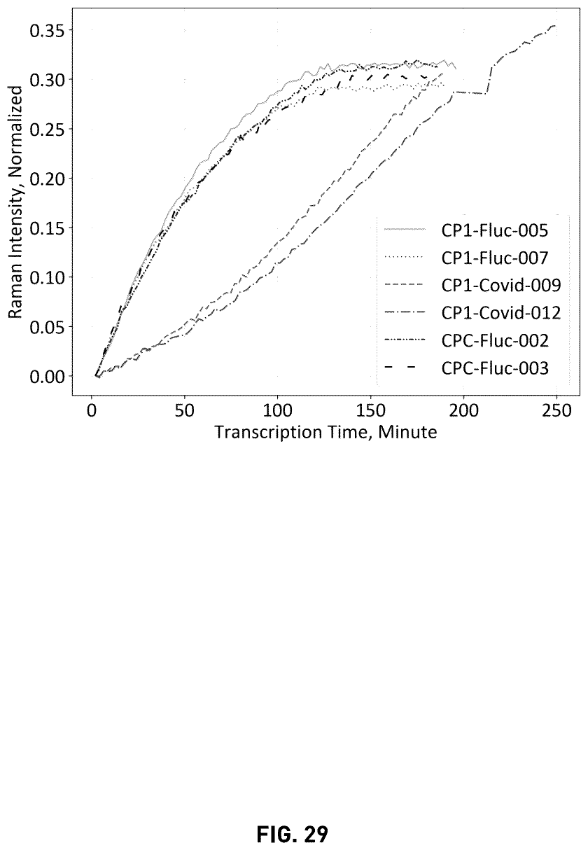

FIG. 29 is a graph illustrating Raman intensity at 990 cm⁻¹ as a function of transcription time. The Raman peak at 990 cm⁻¹ is hypothesized to correspond to the HPO₄⁻² formation during mRNA transcription.

### EXAMPLE 3: MULTIVARIATE MODELLING WITH IN-LINE RAMAN TO PREDICT NTPS CONCENTRATION DURING IVT REACTION

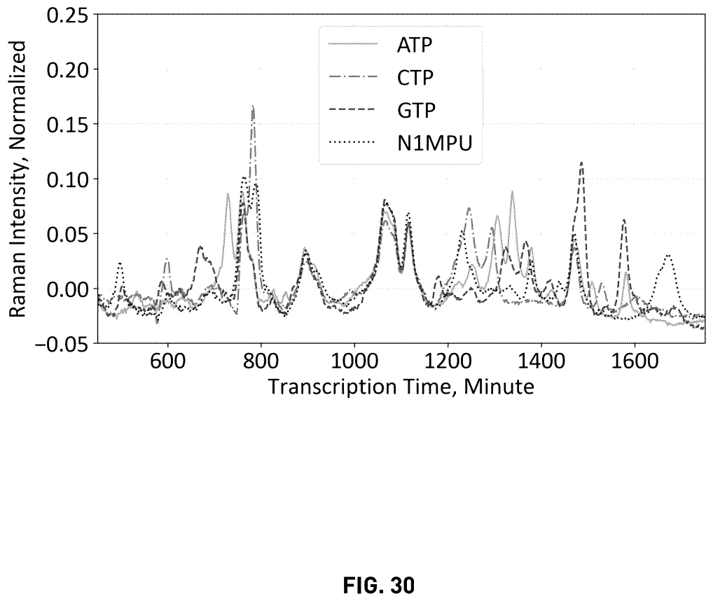

FIG. 30 is a graph illustrating Raman spectra of NTPs obtained by principal component analysis (PCA). Four samples were created for each NTP with 2 mM, 4 mM, 8 mM, and 16 mM concentration. Samples were measured with Raman spectroscopy and PCA was performed to obtain the fingerprint spectrum of each NTP.

TABLE 2
Sample list from CP1 and CPC process used for PLS model development for NTP monitoring.

| | | | | Sample | | | | | |
|---|---|---|---|---|---|---|---|---|---|
| | | Scale, | | Time, | Off-line Concentration, mM | | | | |
| Number | Run ID | ml | Construct | minutes | CTP | N1MPU | GTP | ATP | Used for |
| 1 | CPC-Fluc-001 | 10 | Fluc | 30 | 5.23 | 8.18 | 6.03 | 6.69 | |
| 2 | CPC-Fluc-001 | 10 | Fluc | 60 | 2.78 | 6.50 | 3.82 | 4.67 | |
| 3 | CPC-Fluc-001 | 10 | Fluc | 90 | 1.00 | 5.66 | 2.25 | 3.36 | |
| 4 | CPC-Fluc-001 | 10 | Fluc | 120 | 0.00 | 0.64 | 0.16 | 0.32 | |
| 5 | CPC-Fluc-001 | 10 | Fluc | 150 | 0.00 | 4.65 | 1.22 | 2.33 | |
| 6 | CPC-Fluc-001 | 10 | Fluc | 180 | 0.00 | 4.83 | 1.27 | 2.45 | |
| 7 | CPC-Fluc-002 | 10 | Fluc | 16 | 5.21 | 6.19 | 5.44 | 5.70 | |
| 8 | CPC-Fluc-002 | 10 | Fluc | 30 | 3.97 | 5.50 | 4.33 | 4.74 | |
| 9 | CPC-Fluc-002 | 10 | Fluc | 46 | 2.39 | 3.94 | 2.76 | 3.18 | |
| 10 | CPC-Fluc-002 | 10 | Fluc | 60 | 1.95 | 4.03 | 2.42 | 2.99 | |
| 11 | CPC-Fluc-002 | 10 | Fluc | 76 | 1.60 | 4.26 | 2.19 | 2.92 | |
| 12 | CPC-Fluc-002 | 10 | Fluc | 90 | 1.02 | 4.11 | 1.71 | 2.55 | |
| 13 | CPC-Fluc-002 | 10 | Fluc | 106 | 0.51 | 3.52 | 1.17 | 1.98 | |
| 14 | CPC-Fluc-002 | 10 | Fluc | 120 | 0.17 | 3.66 | 0.93 | 1.87 | |
| 15 | CPC-Fluc-002 | 10 | Fluc | 136 | 0.00 | 3.86 | 0.84 | 1.86 | |
| 16 | CPC-Fluc-002 | 10 | Fluc | 150 | 0.00 | 3.56 | 0.76 | 1.71 | |
| 17 | CPC-Fluc-002 | 10 | Fluc | 166 | 0.00 | 3.99 | 0.85 | 1.91 | |
| 18 | CPC-Fluc-002 | 10 | Fluc | 180 | 0.00 | 3.86 | 0.82 | 1.84 | |

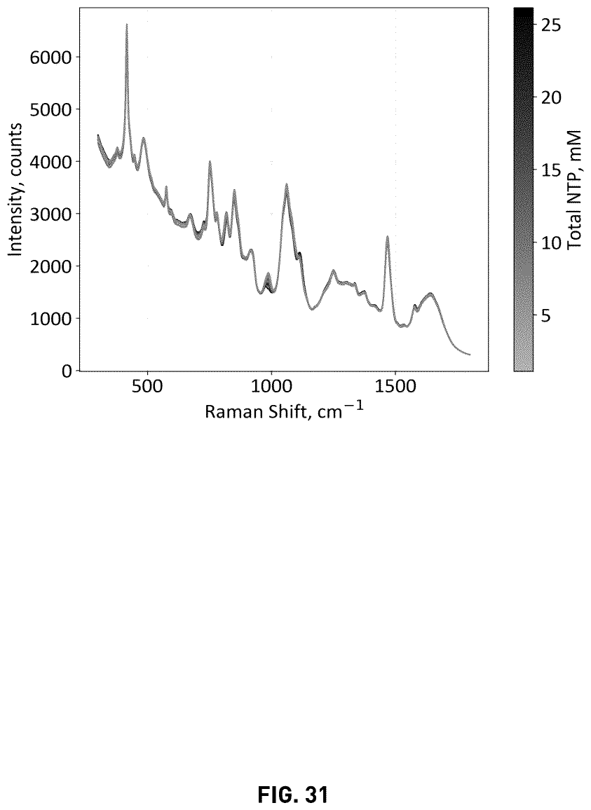

FIG. 31 is a line graph of raw spectral data acquired from IVT runs using an in-line Raman flow cell. The intensity of the raw spectral data does not correlate to total NTP concentration and therefore spectral data needs to be pre-processed for model training.

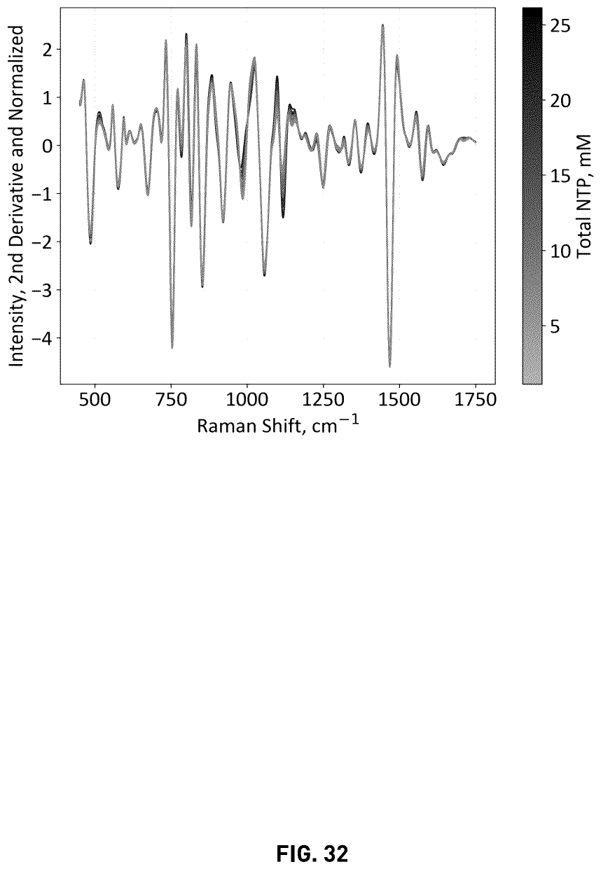

FIG. 32 is a graph illustrating the normalized 2^{nd} derivative of spectral data acquired from IVT runs using an in-line Raman flow cell. After taking the 2^{nd} derivative and normalizing the spectral data, a correlation appears between pre-processed intensity and mRNA concentration.

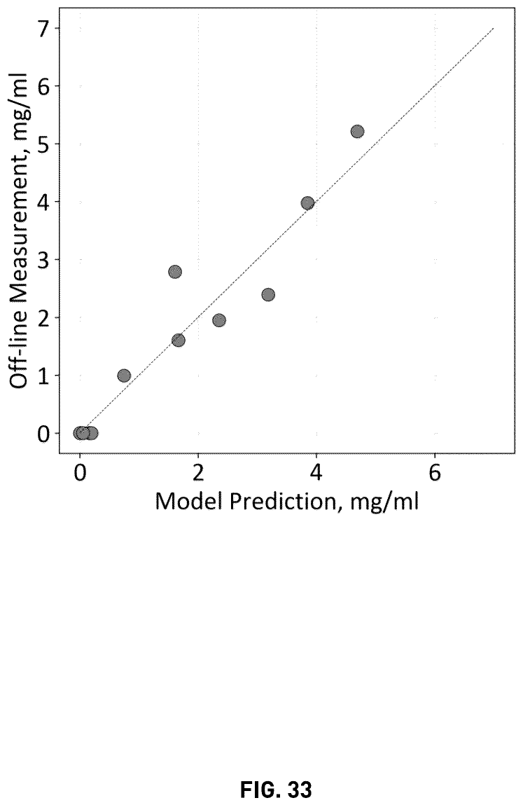

FIG. 33 is a regression plot for training of the PLS model predicting the concentration of CTP. The plot shows the model predictions correlate to the off-line measurements. RMSE of cross-validated dataset=0.43 mM; RMSE of training dataset=0.35 mM; R²=0.95; Q²=0.92.

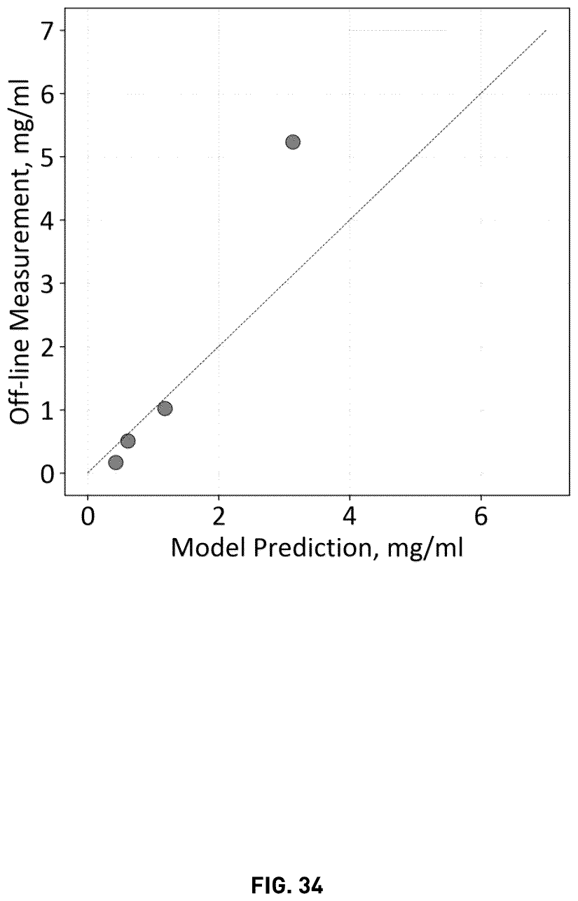

FIG. 34 is a regression plot for validation of the PLS model predicting GTP. This plot shows that the model prediction correlates to the off-line concentration. RMSE of validation dataset=0.9 mM; R² of validation dataset=0.47 (low R2 is due to inadequate number of samples)

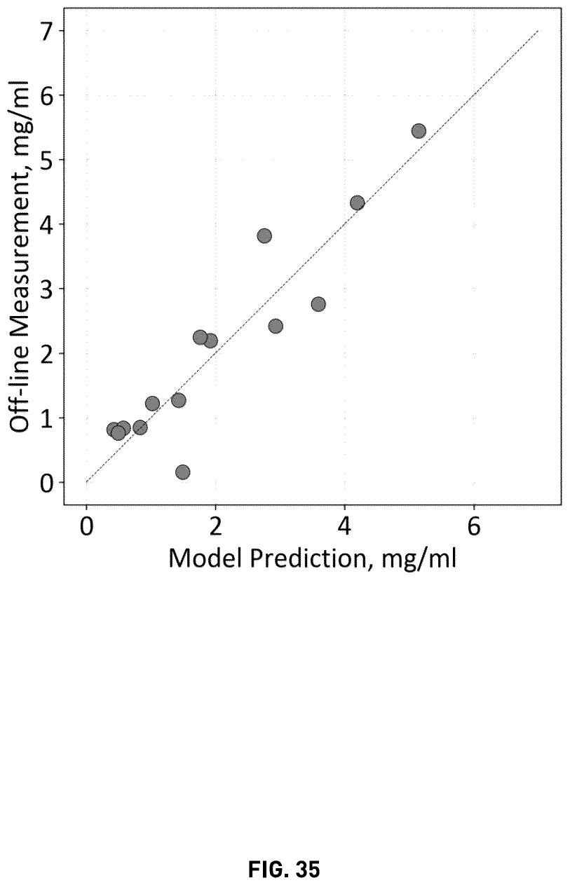

FIG. 35 is a regression plot for training of the PLS model predicting GTP. The plot shows the model predictions correlates to the off-line measurements. RMSE of cross-validated dataset=0.58 mM; RMSE of training dataset=0.48 mM; R²=0.9; Q²=0.84.

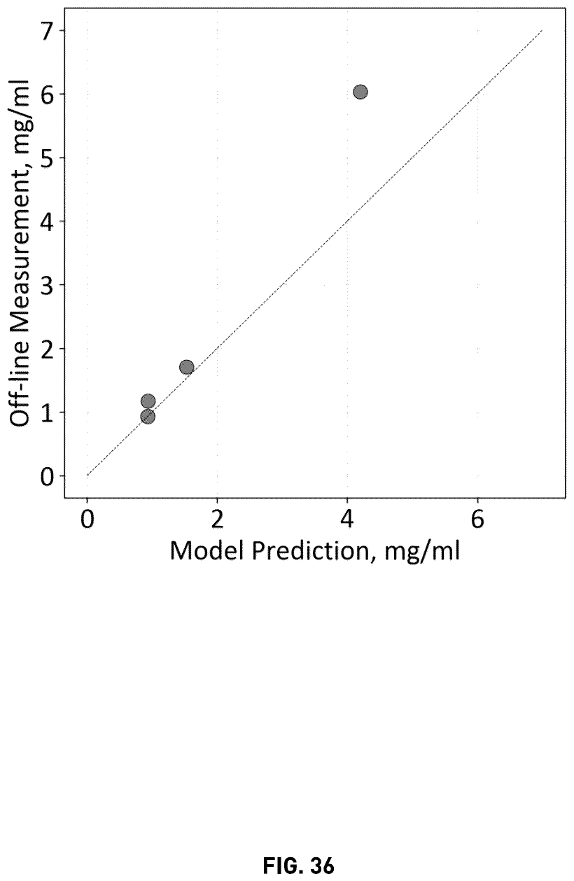

FIG. 36 is a regression plot for validation of the PLS model predicting GTP. This plot shows that the model prediction correlates to the off-line concentration. RMSE of validation dataset=0.93 mM; R² of validation dataset=0.53 (low R2 is due to inadequate number of samples).

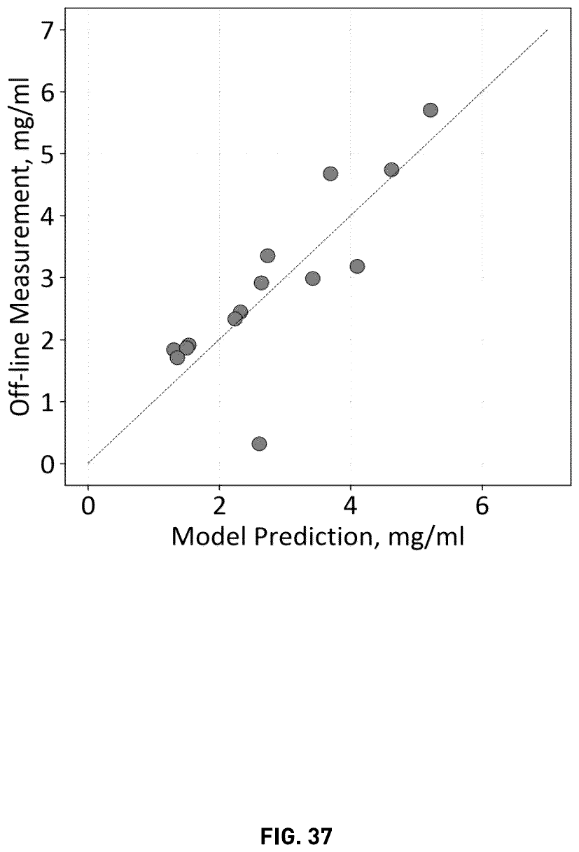

FIG. 37 is a regression plot for training of the PLS model predicting ATP. The plot shows the model predictions correlates to the off-line measurements. RMSE of cross-validated dataset=0.79 mM; RMSE of training dataset=0.65 mM; R²=0.78; Q²=0.57.

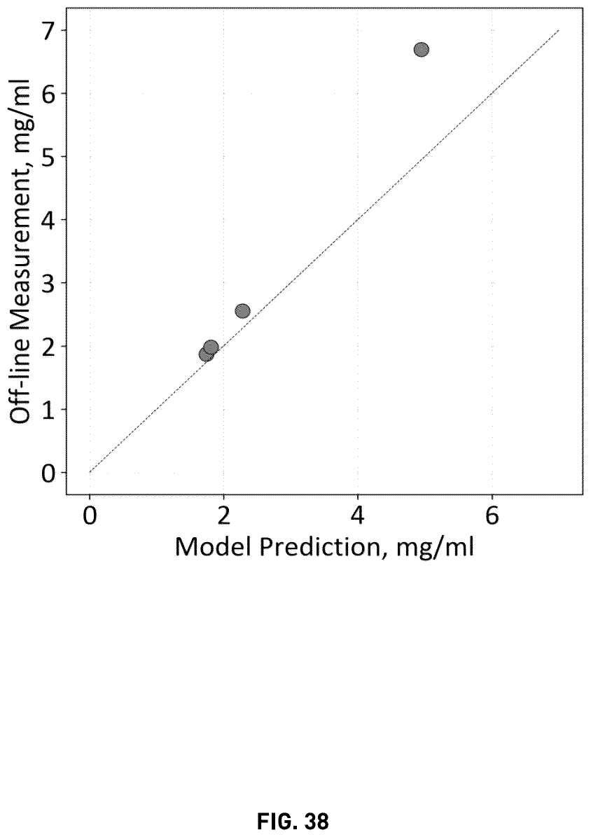

FIG. 38 is a regression plot for validation of the PLS model predicting ATP. This plot shows that the model prediction correlates to the off-line concentration. RMSE of validation dataset=0.89 mM; R² of validation dataset=0.54 (low R2 is due to inadequate number of samples).

### Additional Embodiments

One embodiment of the technology includes an IVT apparatus including a reaction vessel configured for carrying out an RNA in vitro transcription (IVT) reaction, an IVT hold vessel configured to contain a volume of process fluid, one or more optical analytic instruments, and a CQA controller, wherein the apparatus is configured such that the IVT reaction vessel is fluidly connected via a first recirculation line to at least one optical analytic instrument and in fluid communication with the IVT hold vessel via a transfer line, the IVT hold vessel is fluidly connected via a second recirculation line to at least one optical analytic instrument and in fluid communication with a downstream unit operation via a second transfer line, and the CQA controller is configured to (i) receive data from the optical analytic instruments, (ii) monitor an amount of at least one reagent in the IVT reaction vessel and an amount of at least one of RNA product and impurities in the IVT hold vessel, (iii) determine whether a predetermined set of product quality attributes is satisfied, and (iv) execute a set of instructions to release the volume of process fluid from the IVT hold vessel to the downstream unit operation if the predetermined set of product quality attributes is satisfied. In one aspect, the CQA controller includes process analytical tool (PAT) management software, data modeling software, and a process control system (PCS) which may be in the form of a Supervisory Control and Data Acquisition (SCADA) system. In one aspect, the one or more optical analytic instruments is selected from the group consisting of a Raman spectrometer, a UV-VIS spectrometer, a mid-infrared (IR) spectrometer, a near-IR spectrometer, and a refractometer.

In one aspect, the first and second recirculation lines comprise one or more inline sensors and/or flow cells. In one aspect, the first and second transfer lines each comprise a pump and at least one valve configured to receive instructions from the CQA controller. In one aspect, the IVT reaction vessel comprises one or more inlet lines, each in fluid communication with a reservoir comprising at least one reagent of the IVT reaction and each comprising a pump and at least one valve, wherein the at least one valve is configured to receive instructions from the CQA controller. In one aspect, the at least one valve is operated by a proportional-integral-derivative (PID) controller.

In one aspect, the one or more optical analytic instruments is configured to measure an amount of one or more reagents of the IVT reaction. In one aspect, the one or more reagents of the IVT reaction is selected from nucleotides, plasmid DNA template, capping enzyme, RNA polymerase, pyrophosphatase enzyme, and magnesium. In one aspect, the one or more optical analytic instruments is configured to measure an amount of RNA product in the IVT hold vessel. In one aspect, the downstream unit operation is a tangential flow filtration (TFF) operation or a chromatography operation.

One embodiment of the technology includes a TFF apparatus including a TFF filter unit configured to be in fluid communication with an IVT hold vessel and a TFF hold vessel, a TFF hold vessel configured to contain a volume of process fluid and to be in fluid communication with one or more optical analytic instruments via a recirculation line, one or more optical analytic instruments comprising at least one of an inline sensor or inline flow cell, and a CQA controller comprising process analytical tool (PAT) management software, data modeling software, and a process control system (PCS), wherein the apparatus is configured such that in operation the TFF filter unit receives process fluid from the IVT hold vessel, separates RNA product into a product fluid stream, and transfers the product fluid stream to the TFF hold vessel where it recirculates through the at least one inline sensor or flow cell of the one or more optical analytic instruments, and the CQA controller is configured to (i) receive input from the optical analytic instruments, (ii) monitor at least one product attribute of the RNA product, (iii) determine whether a predetermined set of product quality attributes is satisfied, and (iv) execute a set of instructions to release the volume of process fluid from the TFF hold vessel to a downstream unit operation if the predetermined set of product quality attributes is satisfied.

One embodiment of the technology includes a chromatography apparatus comprising a chromatography unit configured to be in fluid communication with an IVT or TFF hold vessel and a chromatography hold vessel, a chromatography hold vessel configured to contain a volume of process fluid and to be in fluid communication with one or more optical analytic instruments via a recirculation line, one or more optical analytic instruments comprising at least one of an inline sensor or inline flow cell, and a CQA controller comprising process analytical tool (PAT) management software, data modeling software, and a process control system (PCS), wherein the apparatus is configured such that in operation the chromatography unit receives process fluid from the IVT hold vessel, separates RNA product into an eluate, and transfers the eluate to the chromatography hold vessel where it recirculates through the at least one inline sensor or flow cell of the one or more optical analytic instruments, and the CQA controller is configured to (i) receive input from the optical analytic instruments, (ii) monitor at least one product attribute of the RNA product, (iii) determine whether a predetermined set of product quality attributes is satisfied, and (iv) execute a set of instructions to release the volume of process fluid from the chromatography hold vessel to a downstream unit operation if the predetermined set of product quality attributes is satisfied.

In one aspect, the one or more optical analytic instruments is selected from the group consisting of a Raman spectrometer, a UV-VIS spectrometer, a mid-infrared (IR) spectrometer, near-IR spectrometer, and a refractometer. In one aspect, the one or more optical analytic instruments is selected from the group consisting of a Raman spectrometer, a UV-VIS spectrometer, a mid-infrared (IR) spectrometer, near-IR spectrometer, and a refractometer. In one aspect, the apparatus comprises a recycling TFF filter unit configured to be in fluid communication with and situated between either the TFF unit of the TFF apparatus or the chromatography unit of the chromatography apparatus and the IVT reaction vessel.

In one aspect, the recycling TFF filter unit is configured to receive a wash fluid from the TFF unit or the chromatography unit. In one aspect, the recycling TFF filter unit is configured to separate at least one reagent of the IVT reaction from the wash fluid and return the at least one reagent to the IVT reaction vessel. In one aspect, the one or more optical analytic instruments comprises a flow cell for inline sampling of the process fluid.

In one aspect, the CQA controller performs chemometrics utilizing offline analytics. In one aspect, the one or more optical analytic instruments includes a Raman spectrometer a mid-IR spectrometer and/or a UV-VIS spectrometer configured to measure a concentration of RNA product in one or more of the IVT reaction vessel, the IVT hold vessel, a TFF hold vessel, or a chromatography hold vessel. In one aspect, the one or more optical analytic instruments includes a Raman spectrometer configured to measure a concentration of impurities in the RNA product in one or more of the IVT reaction vessel, the IVT hold vessel, a TFF hold vessel, or a chromatography hold vessel. In one aspect, the predetermined set of product quality attributes includes a concentration of RNA product and a percentage purity of the RNA

One embodiment of the technology includes a system comprising an IVT apparatus as described herein and one or more of a TFF apparatus and a chromatography apparatus as described herein, wherein the system is configured such that a chromatography apparatus is in fluid communication with the IVT apparatus and a downstream TFF apparatus, wherein the system is configured such that a chromatography unit receives process fluid from an IVT hold vessel, a chromatography hold vessel receives process fluid from the chromatography unit, a TFF filter unit receives process fluid from the chromatography hold vessel, separates RNA product into a product fluid stream, and transfers the product fluid stream to a TFF hold vessel where it recirculates through at least one inline sensor or flow cell of one or more optical analytic instruments, and a CQA controller comprising process analytical tool (PAT) management software, data modeling software, and a process control system (PCS), wherein the CQA controller is configured to (i) receive input from the optical analytic instruments, (ii) monitor at least one product attribute of the RNA product in the TFF hold vessel, (iii) determine whether a predetermined set of product quality attributes is satisfied, and (iv) execute a set of instructions to release the volume of process fluid from the TFF hold vessel to a downstream unit operation if the predetermined set of product quality attributes is satisfied.

In one aspect, the system includes a second TFF apparatus situated between the IVT hold vessel and the chromatography apparatus and configured to receive process fluid from the IVT hold vessel, the second TFF apparatus including a TFF filter unit in fluid communication with the IVT hold vessel and a TFF hold vessel, a TFF hold vessel in fluid communication with one or more optical analytic instruments via a recirculation line, wherein the TFF hold vessel is configured to contain a volume of process fluid, one or more optical analytic instruments comprising at least one of an inline sensor or inline flow cell, and a CQA controller comprising process analytical tool (PAT) management software, data modeling software, and a process control system (PCS), wherein the CQA controller is configured to (i) receive input from the optical analytic instruments, (ii) monitor at least one product attribute of the RNA product in the TFF hold vessel, (iii) determine whether a predetermined set of product quality attributes is satisfied, and (iv) execute a set of instructions to release the volume of process fluid from the TFF hold vessel to the chromatography unit if the predetermined set of product quality attributes is satisfied.

In one aspect, the one or more optical analytic instruments comprises a flow cell for inline sampling of the process fluid. In one aspect, the one or more optical analytic instruments comprises a flow cell for inline sampling of the process fluid. In one aspect, the CQA controller performs chemometrics utilizing offline analytics. In one aspect, the apparatus further comprises a digital twin operably connected to the CQA controllers. In one aspect, the digital twin controls the apparatus. In one aspect, the one or more optical analytic instruments includes a Raman spectrometer a mid-IR spectrometer and/or a UV-VIS spectrometer configured to measure a concentration of RNA product in one or more of the IVT reaction vessel, the IVT hold vessel, a TFF hold vessel, or a chromatography hold vessel. In one aspect, the one or more optical analytic instruments includes a Raman spectrometer configured to measure a concentration of impurities in the RNA product in one or more of the IVT reaction vessel, the IVT hold vessel, a TFF hold vessel, or a chromatography hold vessel. In one aspect, the predetermined set of product quality attributes includes a concentration of RNA product and a percentage purity of the RNA.

One embodiment of the technology includes a method for continuous RNA manufacturing, the method including contacting an initial amount of a linear plasmid DNA template with a reaction mixture in an in vitro transcription (IVT) reaction vessel under conditions suitable for a IVT reaction, monitoring progression of the IVT reaction via data received by a CQA controller from one or more optical analytic instruments in fluid communication with the IVT reaction vessel via a recirculation line, determining, by a first CQA controller, that the reaction has reached a predetermined state, executing, by the first CQA controller, a set of instructions to release a volume of process fluid from the IVT reaction vessel to an IVT hold vessel, receiving, by a second CQA controller, data from one or more optical analytic instruments in fluid communication with the IVT hold vessel via a recirculation line, determining, by the second CQA controller, whether RNA product has reached a predetermined level and percentage purity in the IVT hold vessel, and executing, by the second CQA controller, a set of instructions to release a volume of process fluid from the IVT hold vessel to a downstream unit apparatus if the RNA product has reached the predetermined level and purity.

In one aspect, the method includes downstream unit apparatus is a tangential flow filtration (TFF) apparatus as described herein. In one aspect, the method includes a first tangential flow filtration (TFF) apparatus upstream of the chromatography apparatus and a second TFF apparatus downstream of the chromatography apparatus. In one aspect, the method includes recovering at least one reagent of the IVT reaction mixture and recycling the at least one reagent to the IVT reaction vessel via operation of a TFF unit in fluid communication with and situated between either the downstream TFF apparatus or the downstream chromatography apparatus and the IVT reaction vessel. In one aspect, the at least one reagent including one or more nucleotides, plasmid DNA template, capping enzyme, RNA polymerase, and/or pyrophosphatase enzyme. In one aspect, the one or more nucleotides include guanosine-5′-triphosphate, adenosine triphosphate, cytidine triphosphate, uridine triphosphate, pseudouridine triphosphate, dihydrouridine triphosphate, 4-thiouridine, inosine triphosphate, 7-methylguanosine triphosphate, 2,7-dimethylguanosine triphosphate, and/or 2,2,7-trimethylguanosine triphosphate. In one aspect, the CQA controllers perform chemometrics utilizing offline analytics. In one aspect, the method further includes controlling the CQA controllers by operation of a digital twin.

While the invention herein disclosed has been described by means of specific embodiments and applications thereof, numerous modifications and variations could be made thereto by those skilled in the art without departing from the scope of the invention set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

Various features of a process system may be used independently of, or in combination, with each other. It will be appreciated that a system as disclosed herein may be embodied in different forms and should not be construed as limited to the illustrated embodiments of the figures.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An IVT apparatus comprising
a reaction vessel configured for carrying out an RNA in vitro transcription (IVT) reaction,
an IVT hold vessel configured to contain a volume of process fluid,
one or more in-line optical analytic instruments, wherein at least one of the in-line optical analytic instruments is a Raman spectrometer,
a CQA controller,
wherein the apparatus is configured such that
the IVT reaction vessel is fluidly connected via a first recirculation line to the Raman spectrometer and in fluid communication with the IVT hold vessel via a transfer line,
the IVT hold vessel is fluidly connected via a second recirculation line to the Raman spectrometer and in fluid communication with a downstream unit operation via a second transfer line, and
the CQA controller is configured to (i) receive data from the Raman spectrometer, (ii) monitor an amount of at least one reagent in the IVT reaction vessel and an amount of at least one of RNA product and impurities in the IVT hold vessel, (iii) determine whether a predetermined set of product quality attributes is satisfied, and (iv) execute a set of instructions to release the volume of process fluid from the IVT hold vessel to the downstream unit operation if the predetermined set of product quality attributes is satisfied.

2. The IVT apparatus of claim 1, wherein the CQA controller comprises process analytical tool (PAT) management software, data modeling software, and a process control system (PCS) which may be in the form of a Supervisory Control and Data Acquisition (SCADA) system.

3. The IVT apparatus of claim 1 or 2, wherein the one or more in-line optical analytic instruments includes a second in-line optical analytic instrument selected from the group consisting of a UV-VIS spectrometer, a mid-infrared (IR) spectrometer, a near-IR spectrometer, and a refractometer.

4. The IVT apparatus of any one of claims 1 to 3, wherein the first and second recirculation lines comprise one or more inline sensors and/or flow cells.

5. The IVT apparatus of claim 4, wherein the one or more inline flow cells is a UV variable pathlength flow cell, a Raman flow cell, a mid-IR flow cell, a near-IR flow cell, or an index of refraction flow cell.

6. The IVT apparatus of any one of claims 1 to 5, wherein the first and second transfer lines each comprise a pump and at least one valve configured to receive instructions from the CQA controller.

7. The IVT apparatus of any one of claims 1 to 6, wherein the IVT reaction vessel comprises one or more inlet lines, each in fluid communication with a reservoir comprising at least one reagent of the IVT reaction and each comprising a pump and at least one valve, wherein the at least one valve is configured to receive instructions from the CQA controller.

8. The IVT apparatus of claim 6 or 7, wherein the at least one valve is operated by a proportional-integral-derivative (PID) controller.

9. The IVT apparatus of any one of claims 1 to 8, wherein the one or more optical analytic instruments is configured to measure an amount of one or more reagents of the IVT reaction.

10. The IVT apparatus of claim 9, wherein the one or more reagents of the IVT reaction is selected from nucleotides, plasmid DNA template, capping enzyme, RNA polymerase, pyrophosphatase enzyme, and magnesium.

11. The IVT apparatus of any one of claims 1 to 8, wherein the one or more optical analytic instruments is configured to measure an amount of RNA product in the IVT hold vessel.

12. The IVT apparatus of any one of claims 1 to 11, wherein the downstream unit operation is a tangential flow filtration (TFF) operation or a chromatography operation.

13. A TFF apparatus comprising
a TFF filter unit configured to be in fluid communication with an IVT hold vessel and a TFF hold vessel,
a TFF hold vessel configured to contain a volume of process fluid and to be in fluid communication with one or more optical analytic instruments via a recirculation line,
one or more optical analytic instruments comprising at least one of an inline sensor or inline flow cell, and
a CQA controller comprising process analytical tool (PAT) management software, data modeling software, and a process control system (PCS),
wherein the apparatus is configured such that in operation the TFF filter unit receives process fluid from the IVT hold vessel, separates RNA product into a product fluid stream, and transfers the product fluid stream to the TFF hold vessel where it recirculates through the at least one inline sensor or flow cell of the one or more optical analytic instruments, and
the CQA controller is configured to (i) receive input from the optical analytic instruments, (ii) monitor at least one product attribute of the RNA product, (iii) determine whether a predetermined set of product quality attributes is satisfied, and (iv) execute a set of instructions to release the volume of process fluid from the TFF hold vessel to a downstream unit operation if the predetermined set of product quality attributes is satisfied.

14. A chromatography apparatus comprising
a chromatography unit configured to be in fluid communication with an IVT or TFF hold vessel and a chromatography hold vessel,
a chromatography hold vessel configured to contain a volume of process fluid and to be in fluid communication with one or more optical analytic instruments via a recirculation line,
one or more optical analytic instruments comprising at least one of an inline sensor or inline flow cell, and
a CQA controller comprising process analytical tool (PAT) management software, data modeling software, and a process control system (PCS),
wherein the apparatus is configured such that in operation the chromatography unit receives process fluid from the IVT hold vessel, separates RNA product into an eluate, and transfers the eluate to the chromatography hold vessel where it recirculates through the at least one inline sensor or flow cell of the one or more optical analytic instruments, and
the CQA controller is configured to (i) receive input from the optical analytic instruments, (ii) monitor at least one product attribute of the RNA product, (iii) determine whether a predetermined set of product quality attributes is satisfied, and (iv) execute a set of instructions to release the volume of process fluid from the chromatography hold vessel to a downstream unit operation if the predetermined set of product quality attributes is satisfied.

15. The apparatus of claim 13 or 14, wherein the one or more optical analytic instruments is selected from the group consisting of a Raman spectrometer, a UV-VIS spectrometer, a mid-infrared (IR) spectrometer, near-IR spectrometer, and a refractometer.

16. The apparatus of any one of claims 13 to 15, wherein the apparatus comprises a recycling TFF filter unit configured to be in fluid communication with and situated between either the TFF unit of the TFF apparatus or the chromatography unit of the chromatography apparatus and the IVT reaction vessel.

17. The apparatus of claim 16, wherein the recycling TFF filter unit is configured to receive a wash fluid from the TFF unit or the chromatography unit.

18. The apparatus of claim 17, wherein the recycling TFF filter unit is configured to separate at least one reagent of the IVT reaction from the wash fluid and return the at least one reagent to the IVT reaction vessel.

19. The apparatus of any one of claims 1 to 18, wherein the one or more optical analytic instruments comprises a flow cell for inline sampling of the process fluid.

20. The apparatus of any one of claims 1 to 19, wherein the CQA controller performs chemometrics utilizing offline analytics.

21. The apparatus of any one of claims 1 to 20, wherein the one or more optical analytic instruments includes a Raman spectrometer a mid-IR spectrometer and/or a UV-VIS spectrometer configured to measure a concentration of RNA product in one or more of the IVT reaction vessel, the IVT hold vessel, a TFF hold vessel, or a chromatography hold vessel.

22. The apparatus of any one of claims 1 to 21, wherein the one or more optical analytic instruments includes a Raman spectrometer configured to measure a concentration of impurities in the RNA product in one or more of the IVT reaction vessel, the IVT hold vessel, a TFF hold vessel, or a chromatography hold vessel.

23. The apparatus of any one of claims 1 to 22, wherein the predetermined set of product quality attributes includes a concentration of RNA product and a percentage purity of the RNA.

24. A system comprising an IVT apparatus according to any one of claims 1 to 12 and one or more of a TFF apparatus and a chromatography apparatus according to any one of claims 13 to 18, wherein the system is configured such that a chromatography apparatus is in fluid communication with the IVT apparatus and a downstream TFF apparatus,
wherein the system is configured such that a chromatography unit receives process fluid from an IVT hold vessel, a chromatography hold vessel receives process fluid from the chromatography unit, a TFF filter unit receives process fluid from the chromatography hold vessel, separates RNA product into a product fluid stream, and transfers the product fluid stream to a TFF hold vessel where it recirculates through at least one inline sensor or flow cell of one or more optical analytic instruments, and
a CQA controller comprising process analytical tool (PAT) management software, data modeling software, and a process control system (PCS), wherein the CQA controller is configured to (i) receive input from the optical analytic instruments, (ii) monitor at least one product attribute of the RNA product in the TFF hold vessel, (iii) determine whether a predetermined set of product quality attributes is satisfied, and (iv) execute a set of instructions to release the volume of process fluid from the TFF hold vessel to a downstream unit operation if the predetermined set of product quality attributes is satisfied.

25. The system of claim 24, wherein the system comprises a second TFF apparatus situated between the IVT hold vessel and the chromatography apparatus and configured to receive process fluid from the IVT hold vessel, the second TFF apparatus comprising
a TFF filter unit in fluid communication with the IVT hold vessel and a TFF hold vessel,
a TFF hold vessel in fluid communication with one or more optical analytic instruments via a recirculation line, wherein the TFF hold vessel is configured to contain a volume of process fluid,
one or more optical analytic instruments comprising at least one of an inline sensor or inline flow cell, and
a CQA controller comprising process analytical tool (PAT) management software, data modeling software, and a process control system (PCS), wherein the CQA controller is configured to (i) receive input from the optical analytic instruments, (ii) monitor at least one product attribute of the RNA product in the TFF hold vessel, (iii) determine whether a predetermined set of product quality attributes is satisfied, and (iv) execute a set of instructions to release the volume of process fluid from the TFF hold vessel to the chromatography unit if the predetermined set of product quality attributes is satisfied.

26. The system of claim 24 or claim 25, wherein the one or more optical analytic instruments comprises a flow cell for inline sampling of the process fluid.

27. The system of claim 24 or claim 25, wherein the CQA controller performs chemometrics utilizing offline analytics.

28. The system of claim 24 or claim 25, wherein the apparatus further comprises a digital twin operably connected to the CQA controllers.

29. The system of claim 28, wherein the digital twin controls the apparatus.

30. The system of any one of claims 24 to 29, wherein the one or more optical analytic instruments includes a Raman spectrometer a mid-IR spectrometer and/or a UV-VIS spectrometer configured to measure a concentration of RNA product in one or more of the IVT reaction vessel, the IVT hold vessel, a TFF hold vessel, or a chromatography hold vessel.

31. The system of any one of claims 24 to 29, wherein the one or more optical analytic instruments includes a Raman spectrometer configured to measure a concentration of impurities in the RNA product in one or more of the IVT reaction vessel, the IVT hold vessel, a TFF hold vessel, or a chromatography hold vessel.

32. The system of any one of claims 24 to 31, wherein the predetermined set of product quality attributes includes a concentration of RNA product and a percentage purity of the RNA.

33. A method for continuous RNA manufacturing, the method comprising
acquiring a series of Raman spectra of process fluid of an in vitro transcription (IVT) reaction via an in-line Raman flow cell in fluid communication with an IVT reaction vessel;
transforming the Raman spectra into IVT Critical Quality Attribute (CQA) data;
monitoring progression of the IVT reaction via the CQA data;
determining, based on the IVT CQA data, whether the IVT reaction has reached a predetermined state; and
executing, by a CQA controller, a set of instructions to release a volume of process fluid from the IVT reaction vessel to an IVT hold vessel if the IVT reaction has reacted a predetermined state.

34. The method of claim 33, wherein the transforming comprises normalizing the Raman spectra and determining an intensity at wavelengths between 800-1200 cm⁻¹.

35. The method of claim 34, wherein the wavelengths include one or more of 812 cm⁻¹, 990 cm⁻¹ and 1122 cm⁻¹.

36. The method of any one of claims 33 to 35, wherein the CQA data is mRNA concentration data or NTP concentration data, or both.

37. The method of any one of claims 33 to 36, wherein the method comprises
acquiring spectral data from a second optical analytic instrument in fluid communication with the IVT hold vessel via an in-line flow cell situated in a recirculation line,
transforming the spectral data into Product Critical Quality Attribute (CQA) data;
determining, by a second CQA controller, whether RNA product has reached a predetermined concentration and percentage purity in the IVT hold vessel based on the Product CQA data, and
executing, by the second CQA controller, a set of instructions to release a volume of process fluid from the IVT hold vessel to a downstream unit apparatus if the RNA product has reached the predetermined level and purity.

38. The method of claim 37, wherein the second optical analytic instrument is a UV-VIS spectrometer.

39. The method of any one of claims 33 to 38, wherein the downstream unit apparatus is a tangential flow filtration (TFF) apparatus according to claim 13, claim 15, claim 16, claim 17, or claim 18.

40. The method of any one of claims 33 to 38, wherein the downstream unit apparatus is a chromatography apparatus according to claim 14, claim 15, claim 16, claim 17, or claim 18.

41. The method of claim 40, wherein the method comprises a first tangential flow filtration (TFF) apparatus upstream of the chromatography apparatus and a second TFF apparatus downstream of the chromatography apparatus.

42. The method of any one of claims 39 to 41, wherein the method comprises recovering at least one reagent of the IVT reaction mixture and recycling the at least one reagent to the IVT reaction vessel via operation of a TFF unit in fluid communication with and situated between either the downstream TFF apparatus or the downstream chromatography apparatus and the IVT reaction vessel.

43. The method of claim 42, wherein the at least one reagent comprises one or more nucleotides, plasmid DNA template, capping enzyme, RNA polymerase, and/or pyrophosphatase enzyme.

44. The method of claim 43, wherein the one or more nucleotides include guanosine-5′-triphosphate, adenosine triphosphate, cytidine triphosphate, uridine triphosphate, pseudouridine triphosphate, dihydrouridine triphosphate, 4-thiouridine, inosine triphosphate, 7-methylguanosine triphosphate, 2,7-dimethylguanosine triphosphate, and/or 2,2,7-trimethylguanosine triphosphate.

45. The method of any one of claims 37 to 44, wherein the CQA controllers perform chemometrics utilizing offline analytics.

46. The method of any one of claims 37 to 45, wherein the method further comprises controlling the CQA controllers by operation of a digital twin.
